(19) United States
(12) Patent Application Publication
(10) Pub. No.: US 2017/0336399 A1
(43) Pub. Date: Nov. 23, 2017

(54) NOVEL METHODS AND DEVICES FOR HIGH-THROUGHPUT QUANTIFICATION, DETECTION AND TEMPORAL PROFILING OF CELLULAR SECRETIONS, AND COMPOSITIONS IDENTIFIED USING SAME

(71) Applicants: YALE UNIVERSITY, New Haven, CT (US); THE JOHNS HOPKINS UNIVERSITY, Baltimore, MD (US)

(72) Inventors: Andre LEVCHENKO, West Haven, CT (US); Kshitiz GUPTA, New Haven, CT (US); David D. ELLISON, Baltimore, MD (US); Yasir SUHAIL, Baltimore, MD (US); Junaid AFZAL, Baltimore, MD (US)

(73) Assignees: YALE UNIVERSITY, New Haven, CT (US); THE JOHN HOPKINS UNIVERSITY, Baltimore, MD (US)

(21) Appl. No.: 15/525,328

(22) PCT Filed: Nov. 13, 2015
(86) PCT No.: PCT/US2015/060647

§ 371 Date: May 9, 2017

Related U.S. Application Data
(60) Provisional application No. 62/080,177, filed on Nov. 14, 2014.

(51) Int. Cl. G01N 33/543; G01N 33/557; G01N 33/569; G06F 19/24

(52) U.S. Cl. CPC G01N 33/54366; G06F 19/24; G01N 33/557; G01N 33/56966; G01N 2570/00

(57) ABSTRACT

The present invention relates to the unexpected discovery of methods and devices that can be used for high-throughput precise quantification, detection and/or temporal profiling of cellular secretions. In various embodiments, the methods of the invention allow for high-throughput absolute detection of secretions of cells, identification of the nature of the secreted molecules, and/or the nature of the secreting cells. Further, the present invention includes a device combining microfluidics and antibody printing, wherein the device can be used to detect protein secretion signature of cells in a high-throughput manner. Further, the present invention includes compositions comprising molecules that can be used to reduce cell death and to implement cell-less therapies. Further, the present invention includes a method for training an algorithm to predict temporal profile of cellular secretion.

### CROSS-REFERENCE TO RELATED APPLICATION

The present application is entitled to priority under 35 U.S.C. §119(e) to U.S. Provisional Patent Application No. 62/080,177, filed Nov. 14, 2014, which is hereby incorporated by reference in its entirety herein.

### STATEMENT REGARDING FEDERALLY SPONSORED RESEARCH OR DEVELOPMENT

This invention was made with government support under GM072024 awarded by National Institute of Health. The government has certain rights in the invention.

### BACKGROUND OF THE INVENTION

Cellular phenotypes, including cell secretions, depend on the biochemical stimuli presented by their microenvironment, such as neighboring cells. Secretion is one of the most common, and effective ways of cell-cell communication. Tightly controlled temporal regulation of secretion of each molecular species is necessary to maintain homeostasis and trigger an appropriate response to a biological stimulus. Cellular secretions can be in the form of a sustained release of a molecular species, or in the form of an impulse, an oscillatory wave, or a more complex kinetic profile.

Estimating the dynamics of cellular secretions is necessary to understand intercellular communication. Bone marrow stem cells (BMSCs) are known to provide beneficial effects in many distinct injured tissues, with possibly distinct mechanisms of cytoprotection. Further, owing to the difficulty of precisely measuring cellular secretions, temporal dynamics of most cellular secretions are poorly understood. It is therefore possible that the differential effects may be obtained by not only distinct multidimensional molecular signatures, but also by distinct temporal dynamics of secretions.

Understanding the secretory signatures of cells in various physiological and pathological contexts is of significant clinical importance. These estimates have proven difficult, not only because of the detection limitations for complex solutions, but because cells modulate what they secrete based on their environment. This makes difficult the determination of the protein “cocktail” secreted by cells in any given environment.

Additionally, paracrine signaling in a biological context is also very dynamic in nature. Paracrine signaling involving any molecular species has a distinct temporal nature, potentially with a significant bearing on the target cell types. Beta cells of the islets of Langerhans secrete insulin in a cyclical nature. Macrophages secrete TNFα and IL-1 in response to injury in the form of a peak, followed by a trough elevated from the baseline secretion rates. Obtaining high-throughput kinetic signature profiles of cell secretions is extremely hard and remains an unresolved challenge.

Further, precise measurement of the phenotype as a response to a biological stimulus is difficult. Existing miniaturized cell secretion measurement platforms measure the accumulated secretions starting from the moment cells are introduced into the system. Since most cells tend to be in phenotypically distinct states when they are unattached (rather than attached), it is difficult to precisely measure cell secretions after they are attached. Similarly, response of cells after any externally applied experimental condition (such as treatment with a drug, changes in oxygen tension, and/or binding of a ligand) cannot be easily measured in a precise manner, since the start of the experiment is ill defined, unless both the cells and the experimental conditions are introduced at the same time.

Absolute measurements of cellular secretions is a difficult task. Existing ELISA-based methods to measure protein secretions suffer from many disadvantages: i) it is very difficult to precisely measure cellular secretions in adherent cells in response to a biological stimulus, ii) it is not possible to clearly define timing of measurement since secreting cells cannot be separated from ELISA spots, iii) it is not possible to detect or predict the kinetics of secretions. Currently available technology allows high-throughput sandwich ELISA-based detection of secretions of small number of cells, but does not allow arbitrary definition of contexts, stimuli or environment (in response to which cells may change their secretory profiles), or detection of temporal profiles of secretions. Currently available secretion detection platforms allow only a static secretory profile to be developed, which does not allow for the identification of specific characteristic profiles for cell types, cell states, and cell responses.

There is an urgent need in the art for methods and devices that can be used to detect absolute cellular secretions. Such methods and devices may be used to predict the temporal profiles of secretions in a high-throughput manner. The present invention addresses this need.

### SUMMARY OF THE INVENTION

As described herein, the present invention relates to methods and devices that can be used for high-throughput precise quantification, detection and/or temporal profiling of cellular secretions.

In one aspect, the invention includes a device for the temporal high-throughput measurement of one or more molecules or compounds secreted by a cell using quantitative enzyme linked immunosorbant assay (qELISA), the device comprising an experimental chamber and an observational chamber, wherein the experimental chamber and the observational chamber are separated by a permeable barrier, wherein the permeable barrier is selected so that movement of the one or more molecules or compounds across the permeable barrier is hindered when the observational chamber comprises air and/or is free of liquid. In one embodiment, the device further comprises one or more standardization chambers, one or more experimental chambers, and/or one or more detection chambers. In another embodiment, experimental chamber allows the adhesion of the cell. In yet another embodiment, the observational chamber comprises rows of molecule or compound detection location, wherein each row is arranged transversely to the experimental chamber and comprises an antibody that selectively binds a biological molecule. In still another embodiment, the one or more molecules or compounds secreted by the cell migrate from the experimental chamber to the observational chamber through diffusion based movement.

In another aspect, the invention includes a method of calculating an intensity of a cellular secretion using the device described herein. The method comprises contacting cells with the experimental chamber, exposing the cells to experimental conditions to induce secretion of the one or more molecules or compounds, moving the one or more molecules or compounds from the experimental chamber into the observation chamber, binding the one or more molecules or compounds to one or more molecule or compound detection locations in the observational chamber, and calculating an intensity of the one or more molecules or compounds.

In yet another aspect, the invention includes a method of generating a temporal intensity profile of one or more molecules or compounds secreted from a cell. The method comprises calculating an estimated intensity of the one or more molecules or compounds at a distinct molecule or compound detection location and time based on diffusion of the one or more molecules or compounds to the detection location (g[x,t]), calculating an observed intensity at the detection location due to an adsorption and binding of the one or more molecules or compounds to the molecule or compound detection location at an observed time (s[x,t]), calculating a difference between b) and a) (s[x,t]−g[x,t]) to obtain a loss function, updating the estimated intensity to minimize the loss function, generating the intensity profile for the one or more molecules or compounds at the molecule or compound detection location, and repeating the steps for a plurality of molecule or compound detection locations, thereby training a function minimization algorithm to generate the temporal intensity profile of the one or more molecules or compounds secreted from a cell.

In still another aspect, the invention includes a method of generating a temporal concentration profile of one or more molecules or compounds secreted from a cell. The method comprises calculating an estimated concentration of the one or more molecules or compounds at a distinct molecule or compound detection location and time based on diffusion of the one or more molecules or compounds to the molecule or compound detection location (c[t]), proposing a deviation (d[t]) from the estimated concentration (c[t]+d[t]), calculating an observed concentration at the molecule or compound detection location due to an adsorption and binding of the one or more molecules or compounds to the molecule or compound detection location at an observed time (s[x,t]), calculating a difference between b) and c) (c[t]+d[t]−s[x,t]) to obtain a posterior probability of the deviation, accepting or rejecting the proposed deviation of d[t] based on the ratio of the posterior probability of (d) compared to the estimated concentration, generating the concentration profile for the one or more molecules or compounds at the molecule or compound detection location, and repeating the steps for a plurality of molecule or compound detection location, thereby training a function minimization algorithm to generate the temporal concentration profile of the one or more molecules or compounds secreted from a cell.

In another aspect, the invention includes a method of detecting a secretion, and/or level of secretion, of a molecule or compound by a cell isolated from a subject, the method comprising measuring and determining temporal intensity profile and/or temporal concentration profile of the molecule or compound using the device described herein. In one embodiment, the subject is a mammal. In another embodiment, the cell is an adherent cell selected from the group consisting of fibroblasts, immune cells, cancer cell lines, primary cancer cells, stem cells, progenitor cells, stromal cells, pluripotent stem cells, somatic cells derived from pluripotent stem cells, and somatic cells derived from adult stem cells. In yet another embodiment, the cell is a non-adherent cell. In still another embodiment, the cell is derived from healthy or diseased heart tissue, connective tissue, vasculature, brain tissue, tumor environment and/or metastatic tumor environment. In another embodiment, the cell is derived from a tissue explant that is placed in the experimental chamber from healthy or diseased heart, vasculature, brain, tumor, liver, pancreas, spleen, bone marrow, cartilage, adipose tissue, and/or connective tissue. In yet another embodiment, the cell is pretreated by a stimulus, such as at least one from the group consisting of a drug, cytokine, growth factor, hypoxia, pathogen load, physical, chemical, mechanical, and biological stimulus. In another embodiment, the cell is cultured in a biologically mimicking environment. In yet another embodiment, the cell is co-cultured in a system selected from the group consisting of cancer cell in the presence of immune cells, immune cell in the presence of cancer cells, stem cell in the presence of immune cells, stem cell in the presence of stromal cells, stromal cell in the presence of stem cells, endothelial cell in the response to cancer cells, cancer cell in the response to endothelial cells, and cancer cell in the presence of other cancer cells.

In yet another aspect, the invention includes a method of identifying a cell isolated from a subject, the method comprising measuring and/or determining a temporal intensity profile and/or temporal concentration profile of one or more molecules or compounds using the device described herein, wherein the profiles identify at least one selected from the group consisting of cell type, cell state, such as cell signaling, cell fate, cell age, and/or cell cycle, and cell response to a biological stimuli.

In still another aspect, the invention includes a method of treating a disease or disorder in a subject in need thereof, wherein the treatment is cell-free, the method comprising the steps of: identifying a first temporal intensity profile and/or temporal concentration profile for one or more molecules or compounds secreted by a cell that is used for treating the disease or disorder, wherein the first profiles comprise one or more biological molecules, identifying a second temporal intensity profile and/or temporal concentration profile for one or more molecules or compounds secreted by various cell types used to treat the same disease or disorder, wherein the second profiles comprise one or more biological molecules, and administering to the subject a therapeutically effective amount of the one or more molecules comprised in either the first or the second profiles, wherein the subject is not administered a therapeutically effective amount of the cell. In one embodiment, the cell comprises at least one selected from the group consisting of stem cell that secretes anti-apoptotic factors, stromal cell that secretes multipotency or differentiating factors, immune cell that secretes chemokines that inhibit cancer, immune cell that secretes chemokines that support cancer invasion secreted, and cancer cell that secretes a chemokine that promotes angiogenesis.

In another aspect, the invention includes a method of identifying post-translational modification of secreted molecules from a cell in a specific biological condition, the method comprising measuring and determining the kinetics and temporal profiles of a cell exposed to a specific biological condition using the device described herein. In one embodiment, the modification is selected from the group consisting of glycosylation, salicylic acid decoration, splicing, polymerization and other post translational modifications.

In yet another aspect, the invention includes a composition comprising one or more growth factors selected from the group consisting of VEGF, SDF-1α, FGF8, IGF1, insulin, HGF, EGF, IGF1, and SCF, wherein the composition provides cytoprotection, such as against peroxide, and prevents cellular apoptosis when contacted with a cell. In one embodiment, the composition comprises IGF1, HGF and SDF-1α. In another embodiment, the composition is used to treat or prevent cardiac injury.

In still another embodiment, the invention includes a method of treating a disease or disorder in a subject in need thereof, the method comprising administering to the subject a composition comprising one or more growth factors selected from the group consisting of VEGF, SDF-1α, FGF8, IGF1, insulin, HGF, EGF, IGF1, and SCF. In one embodiment, the composition comprises IGF1, HGF and SDF-1α. In another embodiment, the disease or disorder comprises cardiac injury.

In yet another aspect, the invention includes a composition comprising one or more molecules, wherein the composition preconditions cells with mechanical and hypoxic preconditioning to induce a desired response, such as cell survival, prevention of cell proliferation, cell differentiation, cell multi- or pluri-potency, cell migration, and other cellular phenotypes.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1J are a series of graphs illustrating the Micro qELISA chip, herein synonymous with μFLISA, which precisely measures cell secretion in arbitrary conditions. FIG. 1A: Schematic showing the layout of the cells and detection system. The cells are to the left, separated from the detection system by an array of PDMS pillars. On the right are rows of microarray printed capture antibodies which are used for a subsequent on-chip sandwich immunoabsorbent fluorescent detection of ligands. FIG. 1B: qELISA can determine context-dependent secretion signature of cells. Schematic showing the signature profiles that are possible to detect for each biological context, allowing identification of cell types by their secretions or comparing their secretory phenotype in response to distinct stimuli. FIG. 1C: Precise and absolute determination of the secretory signature cells can be used to create artificial recipes to mimic the paracrine signaling of the cell. These recipes can then be used to mimic the therapeutic effect of cells as a potential replacement to cell therapy. FIG. 1D: The qELISA platform. To the left, solidworks schematic showing the qELISA platform with experimental and standardization chambers combined in a single chip; in the center is shown the qELISA platform with cultured BMSCs and antibody arrays highlighted using a fluorescent dye; to the right are shown a magnified view of a single qELISA chip with a section of the antibody arrays shown magnified further to the right. qELISA platform can allow simultaneous high-throughput comparison of two distinct biological conditions in cells with precise and absolute concentration determination of cell secretions, and cell secretion kinetics. FIG. 1E: Schematic showing the method of fluidically separating the experimental chamber containing cultured cells, and the detection chamber consisting of antibody arrays using hexagonal pillars. Surface tension requires P2 to be significantly higher than P1, allowing for a manageable range of fluidic pressures to allow for fluidic separation of the chambers, allowing for a clean determination of the start time for detection of secretions. FIG. 1F: ComSol simulation of the secretions of cells around the pillars showing little to no shadow effect even 200 μm away from the pillars. FIG. 1G: Comparison of qELISA chip with flow cytometry-based measurements of secretions show a high correlation. FIG. 1H: qELISA platform can determine precise and absolute concentration of cell secretions in distinct biological contexts. Here, secretions of BMSCs were measured after culturing in normoxia, and hypoxia for 18 hours using qELISA platform for a small set of secretions. FIG. 1I: Schematic showing algorithmic prediction of secretory temporal kinetics from the spatial fluorescence information of secretory signature of cells in μFLISA (qELISA). FIG. 1J: Commonly occurring canonical secretion profiles (left), and solved temporal secretory profile in a μFLISA (qELISA) platform for an impulse function, a step function, and single pulses of different shapes. Temporal profiles are solved from simulated spatial distribution of ligands in detection chamber.

FIGS. 2A-2C are flow charts showing predictive computational module to accurately predict the temporal profiles of cellular secretion from static intensity signatures in qELISA platform. This flow chart shows the algorithm to computationally predict temporal profile of cells from intensity profiles of each qELISA snapshot. FIG. 2A: Flow chart of the main steps preparation steps for the sample to be loaded on the qELISA platform and analyzed. FIG. 2B: Flow chart of the main steps of the function minimization algorithm. FIG. 2C: Flow chart of the main steps of the probability sampling algorithm.

FIGS. 3A-3E are a series of graphs showing that the predictive computational module of the present invention successfully predicts various canonical temporal profiles of cellular secretions. qELISA design allows employing the spatial information from intensity profiles to be used to predict temporal profile of secretion with high confidence. Shown are commonly occurring canonical secretion profiles (left in each panel), and predicted qELISA observation at distinct time points (middle in each panel). Also shown is the solved temporal secretory signature of the cells (right in each panel). Computed qELISA observation and predicted temporal profiles from these observations are shown for (FIG. 3A) an impulse pulse, (FIG. 3B) a step function, (FIG. 3C) a single pulse starting with a basal secretory rate, rising and then dipping below the basal rate to return at it again, (FIG. 3D) a single pulse starting with a zero secretory rate and returning to an increased basal rate after a positive overshoot, (FIG. 3E) a single pulse starting with a zero secretory rate and returning to a positive basal rate after exhibiting an increased and then decreased secretory rate. The algorithm predicts commonly occurring secretory profiles with high confidence from qELISA observations.

FIGS. 4A-4F are a series of graph showings that cells exhibit differential secretion dynamics when presented with differing biological contexts. Concentrations of (FIGS. 4A-4B) DKK1, (FIGS. 4C-4D) SDF-1α, (FIGS. 4E-4F) HGF in the secretions by BMSCs cultured respectively in normoxia and hypoxia measured at 6 hours, 12 hours, and 18 hours after start of measurement. The values shown are integral over time for the secreted species diffused to the given distance, in x.

FIGS. 5A-5F are a series of histograms showing that bone marrow stem cells (BMSCs) exhibit differential secretion profiles in response to conditions mimicking oxidative stress following ischemia reperfusion and/or myocardial infarction. Secretion profiles of BMSCs measuring absolute amounts of HGF, VEGF, IGF-1, DDK1, SCF, and IL6 when BMSCs are cultured in (FIG. 5A) normoxia, and in the presence of (FIG. 5B) 1% oxygen, (FIG. 5C) TNFα, (FIG. 5D) conditioned medium from FBCMR cultures, (FIG. 5E) conditioned medium from human induced pluripotent stem (iPSC)-derived cardiomyocytes (iPSCMR), (FIG. 5F) conditioned medium from iPSCMR insulted with peroxide. Ligand concentrations were measured after conditioning, and the concentration indicates time integration of secretion. For FIGS. 5A-5F, see FIG. 16 for detailed statistical representation. BMSCs in distinct physiological contexts mimicking myocardial infarction and reperfusion indicate distinct secretory signatures.

FIGS. 6A-6B demonstrate that BMSC-induced rescue of cardiomyocytes is replicated by reconstituted cocktail of BMSC secretome in the presence of cardiac reperfusion insult. (FIG. 6A) Rescue of human iPSC-CMs post peroxide treatment when conditioned with control or secretions from BMSCs cultured in normoxia, hypoxia, or secretions from BMSCs treated either TNFα or conditioned medium from healthy cardiac fibroblast or human iPSC-CMs, or injured human iPSC-CMs. Also shown are the percentage of rescued human iPSC-CMs when conditioned with reconstituted cocktail containing the precise factors measured in secretions of BMSCs treated with conditioned medium from injured human iPSC-CMs. (FIG. 6B) Calcein-AM live dead stain showing live, dead human iPSC-CMs after treatment with the reconstituted anti-apoptotic cocktail (FIG. 6A).

FIGS. 7A-7C illustrate that MicroELISA chip reveals that CDCs secrete IGF-1, HGF, and SDF-1α in normoxia, but SDF-1α secretion is compromised in hypoxia. (FIGS. 7A-B) Microfluidics-based cell secretion analysis system probed for CDC secretion for 6 hours cultured in normoxia (FIG. 7A), and hypoxia (FIG. 7B). FIG. 7C: Standardization curves show intensities detected by microfluidics-based ELISA system for distinct dosages of recombinant proteins.

FIGS. 8A-8F are a series of graphs showing that high-throughput microspotting-based screening reveals cytoprotective factors reducing reperfusion-based cell death in CDCs. FIG. 8A: Schematic showing the method used to detect cell apoptosis in a high-throughput protein microspotting array. Cells cultured on protein+gelatin microspots were treated with 500 μM H₂O₂ for 30 minutes, fixed and labeled with propidium iodide and analyzed using microscopy. Factors that reduce apoptosis significantly compared to control were iteratively combined, till further reduction in apoptosis was not achieved. In certain embodiments, the objective was to find a cocktail of minimum number of factors that reduces peroxide induced apoptosis of CDCs maximally. FIG. 8B: Representative example of a microspot high-throughput array with a distinct condition in each row, also labeled with IgG conjugated with Alexa 488 for visualization. CDCs treated with 500 μM H₂O₂ showed high PI staining, while those untreated showed little cell death. Intermediate concentration of H₂O₂ showed intermediate level of PI staining; Blue=DAPI; Red=PI. FIG. 8C: High-throughput screen of 30 microspotted factors exhibited cytoprotective effects of various species. FIG. 8D: Quantitative analysis of CDCs preconditioned with biochemical factors, and treated with 500 uM H₂O₂ for 30 min showed decreased apoptosis after preconditioning with IGF1, HGF, TNFα, FGF8, SDF-1α, and insulin. Positive control refers to 0 μM H₂O₂, while negative control refers to 500 μM H₂O₂ treatment. FIGS. 8D-8E: Flow cytometry analysis of PI staining in CDCs cultured in the presence of iterative addition to selected optimal cytoprotective pairs in FIG. 8D. FIG. 8F: Quantitative analysis of PI+ peroxide treated CDCs preconditioned with iterative combination of biochemical factors till further significant decrease in PI+ staining does not occur. Positive control refers to 0 μM H₂O₂, while negative control refers to 500 μM H₂O₂ treatment.

FIGS. 9A-9E are a series of figures and graphs showing that a combination of minimal biochemical cocktail with environmental factors can create a comprehensive preconditioning strategy to prevent peroxide-induced CDC apoptosis. FIG. 9A: WST-8 assay shows that CDC survive most after peroxide treatment on polyacrylamide gel with rigidity matching myocardium, 14 kPa. FIG. 9B: Flow cytometry-based PI staining analysis of CDCs cultured on substrata with differing rigidities, and on control surface and treated with peroxide show maximal decrease in cell death on rigidity matching myocardium. FIG. 9C: Flow cytometry analysis showing PI staining is reduced in CDCs preconditioned with minimal biochemical cocktail and simultaneously cultured on substratum with rigidity matching myocardium. FIG. 9D: Quantification of results from FIG. 9 C. FIG. 9E: Flow cytometry analysis showing PI staining is reduced in CDCs preconditioned with minimal biochemical cocktail and simultaneously cultured in the presence of hypoxia emulation using 1% 02, 5% CO2, balance nitrogen. FIG. 9F: Quantification of results from FIG. 9E. FIG. 9G: Flow cytometry-based analysis show that combination of minimal biochemical cocktail, rigidity matching myocardium, and hypoxic preconditioning together further reduce PI staining in peroxide treated CDCs more than individual factors, or pairwise combination of factors.

FIGS. 10A-10C are a series of images and histograms demonstrating that comprehensive preconditioning of CDCs prior to injection in a rat model of ischemia reperfusion and perfusion prevents cell death. FIG. 10A: Photographic image of a reperfused rat heart 1 hour after infarction by ligating the anterior coronary artery shows a large area of injured tissue near the apex. FIG. 10B: Representative bioluminescence images of freshly removed rat hearts 2 days after injecting with CDC-lv-luciferase, 30 minutes after peritoneal injection of luciferin in rats. FIG. 10C: Quantitative analysis of bioluminescence radiance in B show low bioluminescence in the infarcted and re-perfused heart injected with untreated CDCs compared to preconditioned CDCs. Untreated CDCs injected in uninfarcted hearts show high radiance compared to infarcted and reperfused heart. N=3.

FIGS. 11A-11D are a series of graphs demonstrating BMSCs show differential secretion dynamics when presented with different biological contexts. FIGS. 11A-11B: Concentrations and predicted kinetics of secreted SDF-1α by BMSCs in normoxia and hypoxia show very distinct profiles. FIG. 11A: Shown are detected (solid lines) and computed (dotted lines) concentrations at different distances from experimental chamber detected in normoxia, and hypoxia at different locations in a μFLISA platform 6 hours (bottom lines), 12 hours (middle lines), and 18 hours (topmost lines) after start of the experiment; Squared standard error (SSE) values are 0.035 for hypoxia, and 0.061 for normoxia. FIG. 11B: Computed family of predicted temporal profiles of SDF-1α secretion in normoxia, and hypoxia; family of curves were obtained by varying the key parameter by 50% around the value that provides the best fit in FIG. 11A. FIGS. 11C-11D: Concentrations and predicted kinetics of secreted HGF by BMSCs in normoxia and hypoxia show very distinct profiles. FIG. 11C: Shown are detected (solid lines) and computed (dotted lines) concentrations at different distances from experimental chamber detected in normoxia, and hypoxia at different locations in a μFLISA platform 6 hours (bottom lines), 12 hours (middle lines), and 18 hours (topmost lines) after start of the experiment; Squared standard error (SSE) values are 0.035 for hypoxia (FIG. 11D) and 0.061 for normoxia. (FIG. 11D) Computed family of predicted temporal profiles of HGF secretion in normoxia, and hypoxia; family of curves were obtained by varying the key parameter by 50% around the value that provides the best fit in FIG. 11C.

FIGS. 12A-12F are a series of graphs showing precise secretory signatures of BMSCs in the context of injured myocardium can be mimicked to create a cytoprotective cocktail. FIG. 12A is a panel of graphs showing flow cytometry dot plots of Annexin-V and PI staining of hiPSC-CMs treated with secretions from BMSC conditioned with factors listed in FIGS. 5A-5F, and an artificial biochemical cocktail precisely mimicking the secretory signature in FIG. 5F. FIG. 12B shows quantification of hiPSC-CM death by Annexin V/PI based flow cytometry in the presence of 500 μM H2O2 after treatment with factors present in FIG. 5A-5F, and biochemical cocktail mimicking FIG. 5F. FIG. 12C shows DCF-DA intensity in hiPSC-CMs in the presence of 500 μM H2O after treatment with factors mimicking the biochemical cocktail in concentration, and kinetics; untreated cells, and cells not conditioned with cocktail shown as controls; n=4 with >1000 cells. FIG. 12D shows Caspase-3 activation measured in hiPSC-CMs in the conditions above after 30 minutes of treatment with 500 μM H2O2; z-VAD-fmk treated cells shown as positive control; n=3 experiments. FIG. 12E hiPSC-CM death by Annexin V/PI based flow cytometry in the presence of 500 μM H2O2 after treatment with factors mimicking the biochemical cocktail in concentration, and kinetics; untreated cells, and cells not conditioned with cocktail shown as controls; n=3 with >10000 cells. FIG. 12F is a schematic showing that biological context induces cells to secrete factors constituting a unique soluble biochemical signature, and this signature is recognized by the target cells to trigger a desired phenotype.

FIGS. 13A-13D are a series of graphs showing. μFLISA platform facilitates high throughput absolute measurements of cellular secretion time course in response to an arbitrary biological stimulus. FIG. 13A shows ComSol simulation of the μFLISA chip shows that in physiological diffusion rates, saturation is not reached in the whole width of the chip for at least 12 hours, allowing for a high dynamic range for determination of temporal kinetics of the secretions. FIG. 13B shows ComSol simulation of the secretions of cells around the pillars showing little to no shadow effect even 200 μm away from the pillars. FIG. 13C shows standardization curves with 6 capture antibodies printed onto the glass slide, allowing for an absolute determination of protein secretion. In addition to the typical standards used in sandwich ELISA, μFLISA consists of microspots with BSA to account for non specific binding, and PBS to account for carryover of antibodies by microneedle. FIG. 13D is a graph showing on-chip mini standards in each μFLISA platform to calibrate and minimize inter-platform variations; the mini-standards are probed and measured with predetermined concentrations of ligands.

FIGS. 14A-14E are a series of graphs showing computational modeling of spatial distribution of secretory molecules in μFLISA platform. Commonly occurring canonical secretion profiles (left), and computed spatial concentration distribution (right) of a given molecule in μFLISA platform. Predictions are shown for secretion profile of (FIG. 14A) an impulse function, (FIG. 14B) a step function, (FIG. 14C) a single pulse starting and returning at the same concentration, (FIG. 14D) a single pulse ending in a concentration higher than the basal level, (FIG. 14E) a single pulse ending in a concentration higher than the basal level after a trough. Solved spatial distributions of secreted molecule are shown at different time intervals.

FIGS. 15A-15D are a series of graphs showing concentrations and predicted kinetics of secreted DKK1 by BMSCs in normoxia and hypoxia have very distinct profiles. FIGS. 15A-15B: Detected (solid lines) and computed (dotted lines) concentrations at different distances from experimental chamber detected in normoxia (FIG. 15B) and hypoxia (FIG. 15A) at different locations in a μFLISA platform 6 hours, 12 hours, and 18 hours after start of the experiment; Squared standard error (SSE) values are 0.237 for hypoxia and 0.050 for normoxia. The bottom panel shows computed family of predicted temporal profiles of DKK1 secretion in normoxia (FIG. 15D), and hypoxia (FIG. 15C); family of curves were obtained by varying the key parameter by 50% around the value that provides the best fit.

FIGS. 16A-16G are a series of graphs showing secretions of BMSCs are uniquely determined by the biological context. BMSCs exhibit different secretory signatures in the presence of (FIG. 16A) normoxia, (FIG. 16B) hypoxia, (FIG. 16C) TNFα, (FIG. 16D) medium conditioned by cardiac fibroblasts, (FIG. 16E) medium conditioned by hiPSC-CMs, and (FIG. 16F) medium conditioned by hiPSC-CMs insulted with peroxide to mimic ischemia reperfusion injury. FIG. 16G is a combined bar graph showing the biochemical signature of BMSC secretion in the context of impaired myocardium.

FIGS. 17A-17D are a series of graphs showing concentrations within the μFLISA detection chamber of factors detected in the BMSCs secretion in response to conditioning of medium from injured hiPSC-CMs for (FIG. 17A) HGF, (FIG. 17B) IGF-1, and (FIG. 17C) SDF-1α. FIG. 17D shows dosages to mimic the factors present in the biochemical cocktail by average (dashed lines), and by matching the computed dynamics (solid lines); Factors were changed every 1 hour for 18 hours before insulting the conditioned hiPSC-CMs with 500 μM peroxide. Mathematical modeling was used to generate dynamic temporal secretory profiles from spatial μFLISA fluorescence information.

### DETAILED DESCRIPTION OF THE INVENTION

The present invention relates to the unexpected discovery of methods and devices that can be used for high-throughput, precise quantification, detection and/or temporal profiling of cellular secretions. In various embodiments described herein, the methods of the invention allow for high-throughput absolute detection of cellular secretions, identification of the nature of the secreted molecules, and/or identification of the nature of the secreting cells.

The present invention further includes a device combining microfluidics and antibody printing, wherein the device can be used to detect protein secretion signature of cells in a high-throughput manner.

The present invention further includes compositions comprising one or more molecules, wherein the compositions reduce cell death and can be used in cell-less therapies.

The present invention further includes an algorithm that allows for the prediction of temporal profile of cellular secretion.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, specific materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more specifically ±5%, even more specifically ±1%, and still more specifically ±0.1% from the specified value, as such variations are appropriate to perform the disclosed methods.

The term “ameliorating” or “treating” means that the clinical signs and/or the symptoms associated with the disease or disorder are lessened as a result of the actions performed. The signs or symptoms to be monitored will be characteristic of a particular disease or disorder and will be well known to the skilled clinician, as will the methods for monitoring the signs and conditions.

As used herein the term “amount” refers to the abundance or quantity of a constituent in a mixture.

As used herein, the term “amplicon” or “PCR products” or “PCR fragments” or “amplification” products refers to extension products that comprise the primer and the newly synthesized copies of the target sequences.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or VH), VHH domains, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “antibody microspot” as used herein refers to a molecule detection location that comprises a detection reagent, such as antibodies, to bind the secreted molecule or compound under observation. The antibody microspot can be between about 0.1 μm to about 100 μm is size. An array comprises a plurality of microspots. An individual microspot may comprise one or more antibodies to one or more secreted molecules or compounds. In one embodiment, the array comprises a plurality of microspots comprising antibodies to one or more secreted molecules or compounds.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, an antigen need not be encoded solely by a full length nucleotide sequence of a gene. The present invention includes, but is not limited to, the use of partial nucleotide sequences of more than one gene and that these nucleotide sequences are arranged in various combinations to elicit the desired immune response. Moreover, an antigen need not be encoded by a “gene” at all. An antigen can be generated synthesized or can be derived from a biological sample. Such a biological sample can include, but is not limited to a tissue sample, a tumor sample, a cell or a biological fluid.

The term “cancer” as used herein, includes any malignant tumor including, but not limited to, carcinoma, sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof.

The term “concentration” refers to the abundance of a constituent divided by the total volume of a mixture. The term concentration can be applied to any kind of chemical mixture, but most frequently it refers to solutes and solvents in solutions.

The term “experimental condition” refers to conditions that induce a cell to secrete one or more molecules or compounds.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The phrase “loss function” refers to a quantification of a loss associated to an error(s) committed while estimating a parameter. In one embodiment, the loss function is a difference between an observed and an estimated parameter, such as intensity or concentration.

The term “measuring” according to the present invention relates to determining the amount or concentration, preferably semi-quantitatively or quantitatively. Measuring can be done directly and/or indirectly.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “permeable barrier” as used herein, refers to a barrier between the experimental and observational chambers that may be permeable, such as permeable to specific fluids, gases, molecules and/or compounds. In some embodiments, applying hydrostatic pressure to either the experimental chamber or the observational chamber can create increased permeability of the barrier to the specific fluid, gas, molecule and/or compound.

The term “pillar” as used herein, refers to a permeable barrier between the experimental and observational chambers. A plurality of pillars can be used to create a barrier with gaps between the pillars that creates a surface tension between the two chambers when one chamber has liquid and the other has air.

The term “polynucleotide” includes cDNA, RNA, DNA/RNA hybrid, antisense RNA, siRNA, miRNA, snoRNA, genomic DNA, synthetic forms, and mixed polymers, both sense and antisense strands, and may be chemically or biochemically modified to contain non-natural or derivatized, synthetic, or semisynthetic nucleotide bases. Also, included within the scope of the invention are alterations of a wild type or synthetic gene, including but not limited to deletion, insertion, substitution of one or more nucleotides, or fusion to other polynucleotide sequences.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

A “primer” is an oligonucleotide, usually of about 15, 20, 25, 30, 35, 40, 45 or 50 nucleotides in length, that is capable of hybridizing in a sequence specific fashion to the target sequence and being extended during the PCR.

The terms “quantitative enzyme linked immunosorbant assay,” “qELISA,” “microfluidic fluorescence linked immunoabsorbent assay,” or “μFLISA” are used interchangeably herein and refer to a quantitative assay that measures multiple properties of cell secretions, such as concentration, rate of secretion, etc.

As used herein, the terms “reference” or “control” are used interchangeably, and refer to a value that is used as a standard of comparison.

The term “RNA” as used herein is defined as ribonucleic acid.

The term “sample” or “biological sample” refers to a sample obtained from an organism or from components (e.g., cells) of an organism. A “sample” or “biological sample” as used herein means a biological material from a subject, including but is not limited to organ, tissue, exosome, blood, plasma, saliva, urine and other body fluid. A sample can be any source of material obtained from a subject.

A “subject” or “patient” as used therein may be a human or non-human mammal. Non-human mammals include, for example, livestock and pets, such as ovine, bovine, porcine, canine, feline and murine mammals. In certain embodiments, the subject is human.

The phrase “temporal concentration profile” as used herein refers to a concentration of one or more secreted molecules or compounds at a molecule or compound detection location as measured by calculating an estimated intensity of the one or more molecules or compounds at the distinct detection location and time based on diffusion of the one or more molecules or compounds to the detection location (g[x,t]), calculating an observed intensity at the detection location due to an adsorption and binding of the one or more molecules or compounds to the detection location at an observed time, calculating a difference between the observed intensity and the estimated intensity (s[x,t]−g[x,t]) to obtain a loss function, updating the estimated intensity to minimize the loss function, generating the intensity profile for the one or more molecules or compounds at the detection location, and repeating the steps for a plurality of detection locations.

The term “temporal intensity profile” as used herein refers to a binding intensity of one or more molecules or compounds to a molecule or compound detection location as measured by calculating an estimated concentration of the one or more molecules or compounds at a distinct the detection location and time based on diffusion of the one or more molecules or compounds to the detection location (c[t]), proposing a deviation (d[t]) from the estimated concentration (c[t]+d[t]), calculating an observed concentration at the detection location due to an adsorption and binding of the one or more molecules or compounds to the detection location at an observed time (s[x,t]), calculating a difference between the proposed deviation from the observed concentration and the observed concentration (c[t]+d[t]−s[x,t]) to obtain a posterior probability of the deviation, accepting or rejecting the proposed deviation of d[t] based on the ratio of the posterior probability of compared to the estimated concentration, generating the concentration profile for the one or more molecules or compounds at the detection location, and repeating the steps for a plurality of detection locations.

The term “therapeutic” as used herein means a treatment and/or prophylaxis. A therapeutic effect is obtained by suppression, remission, or eradication of a disease state.

As used herein, to “treat” means reducing the frequency with which symptoms of a disease, disorder, or adverse condition, and the like, are experienced by a subject.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

As used herein, “10% greater” refers to expression levels that are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween, than a control or a reference.

As used herein, “10% lower” refers to expression levels that are at least 10% or more, for example, 20%, 30%, 40%, or 50%, 60%, 70%, 80%, 90% lower or more, and/or 1.1 fold, 1.2 fold, 1.4 fold, 1.6 fold, 1.8 fold, 2.0 fold lower or more, and any and all whole or partial increments therebetween, than a control or a reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2, 7, 3, 4, 5, 5.3, and 6. This applies regardless of the breadth of the range.

### DESCRIPTION

The present invention relates to the discovery of methods and devices that can be used for high-throughput precise quantification, detection and/or temporal profiling of cellular secretions.

Further, the invention relates to a novel technique combining microfabrication and microprinted antibodies for sandwich fluorescent immunoassay. This technique allows for absolute measurement of secretions of cells in any given biological context, as well as estimation of the temporal kinetics of cellular secretions.

The present invention includes a novel quantitative ELISA platform, herein synonymously referred to as (qELISA) or microfluidic fluorescence linked immunoabsorbent assay (μFLISA), that combines microfluidics and antibody printing, to detect the protein secretion signature of cells in a high-throughput manner, while also capturing the kinetics of cell secretions. In addition, the qELISA platform of the present invention allows for precise control of the timing of experiments on adherent as well as non-adherent cells.

The device of the present invention combines the strengths of protein microprinting and microfluidics to understand cell secretions of adherent cells in a high-throughput manner and obtain an estimate of the secretory dynamics. Protein microprinting allows for high-throughput ELISA-based measurements, while microfluidics offers methods to facilitate movement of secreted molecules. Since in a microfluidic device movement of molecules is restricted to diffusion, the device described herein allows for the distance travelled by a molecule to be decoded for its time stamp. Taking advantage of this property, the qELISA platform of the present invention allows for the high-throughput detection of cellular secretions, the measurement after a variety of biological stimulations, and also the estimation of the kinetics of cellular secretions. The device of the present invention thus allows for determining a secretory signature for the cellular response to an arbitrary biochemical stimulus.

The device offers a unique advantage by using microfluidics. Fluid flow is laminar in nature, allowing for diffusion to be the only way for spatial movement of molecules. Diffusion displacement of a molecule in the absence of flow encodes its temporal history. Therefore spatial information can be translated into temporal/historical information.

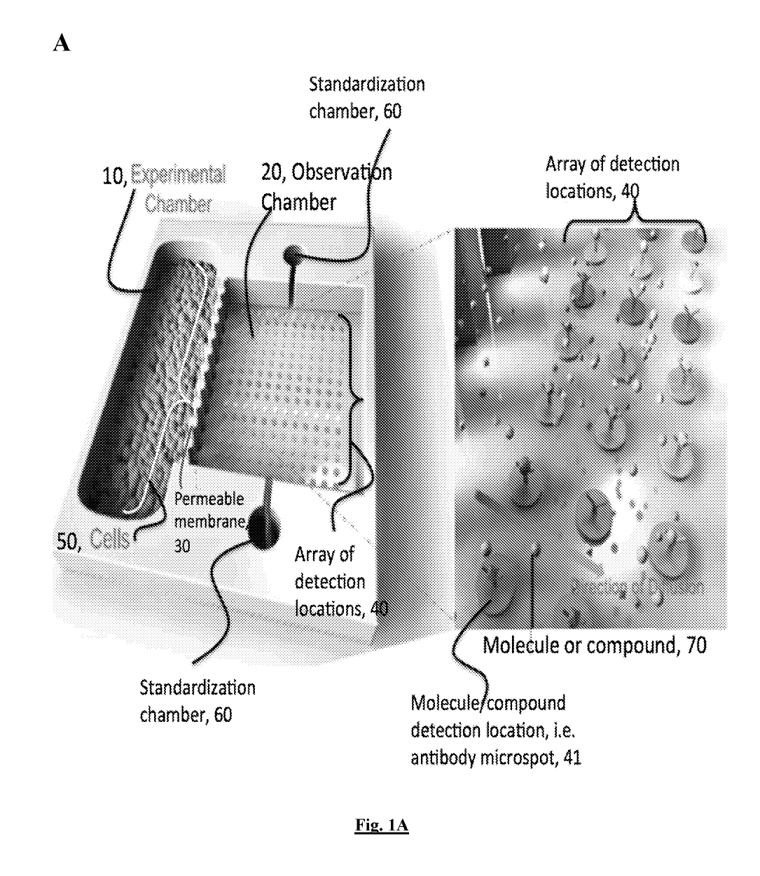

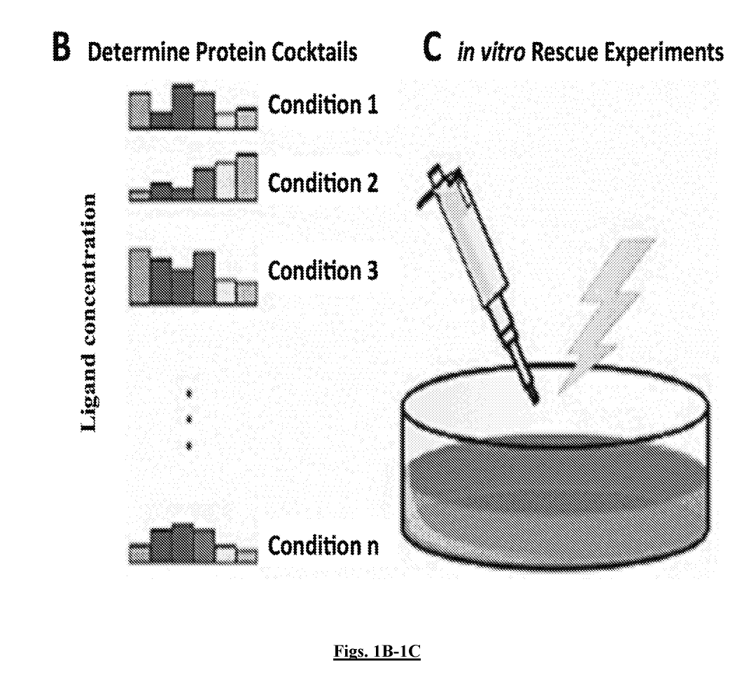

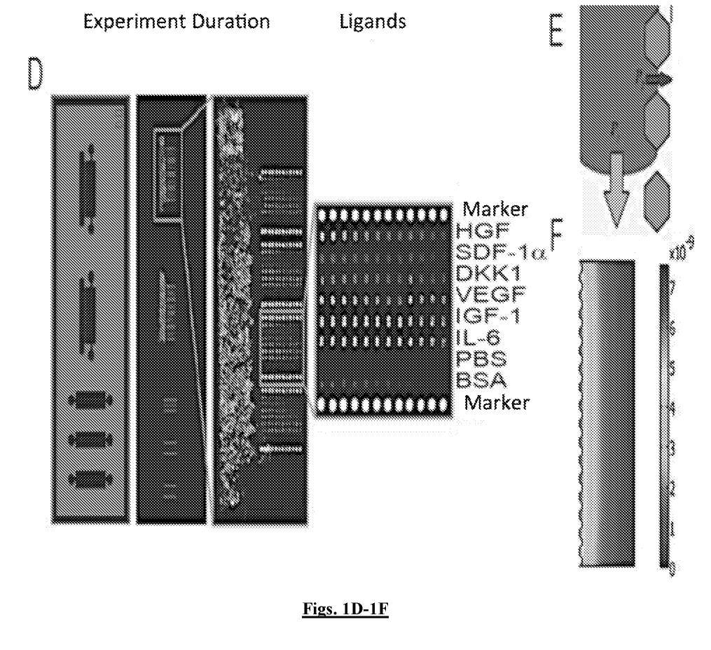

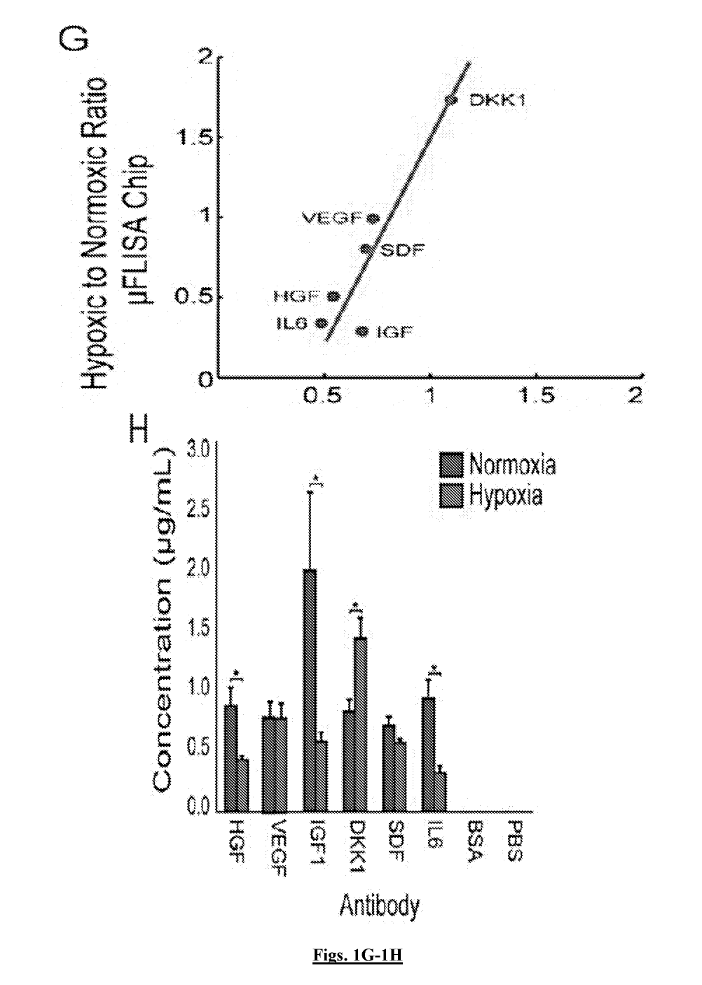

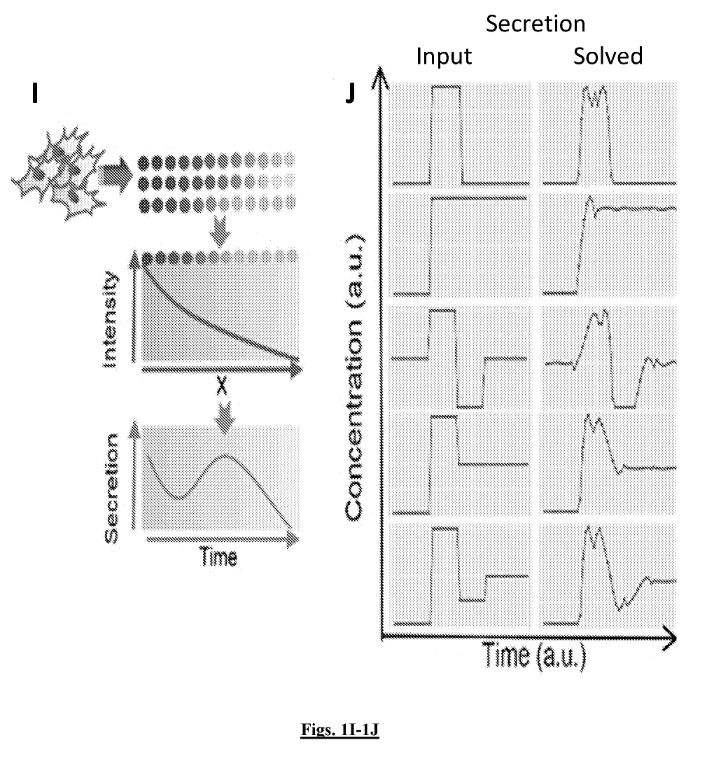

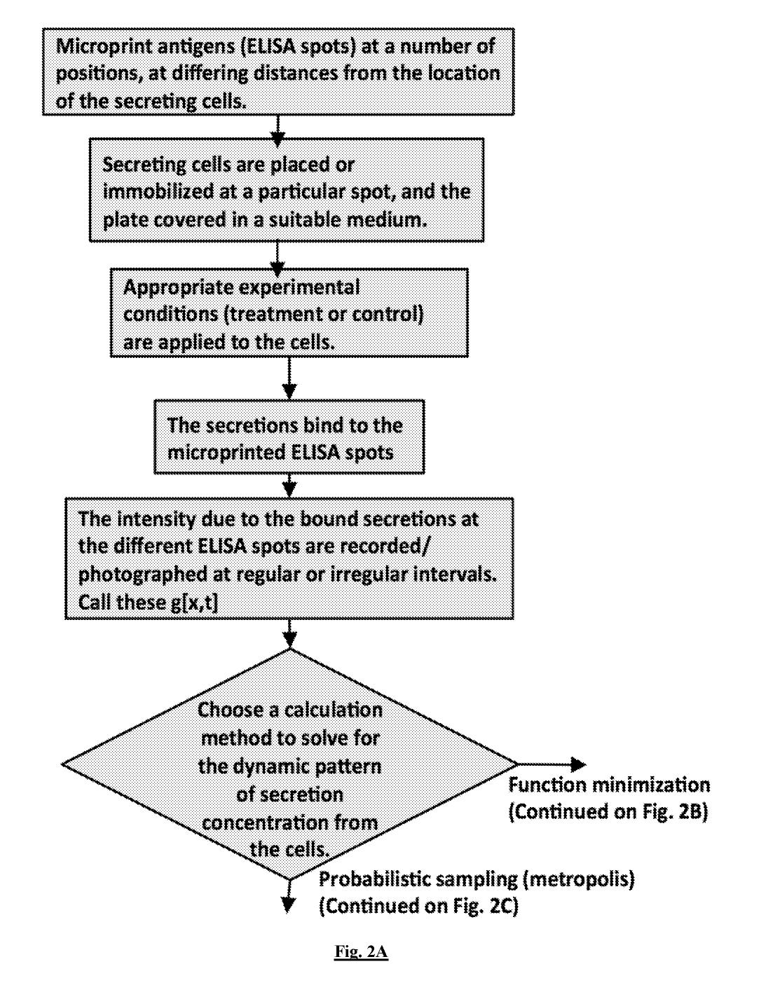

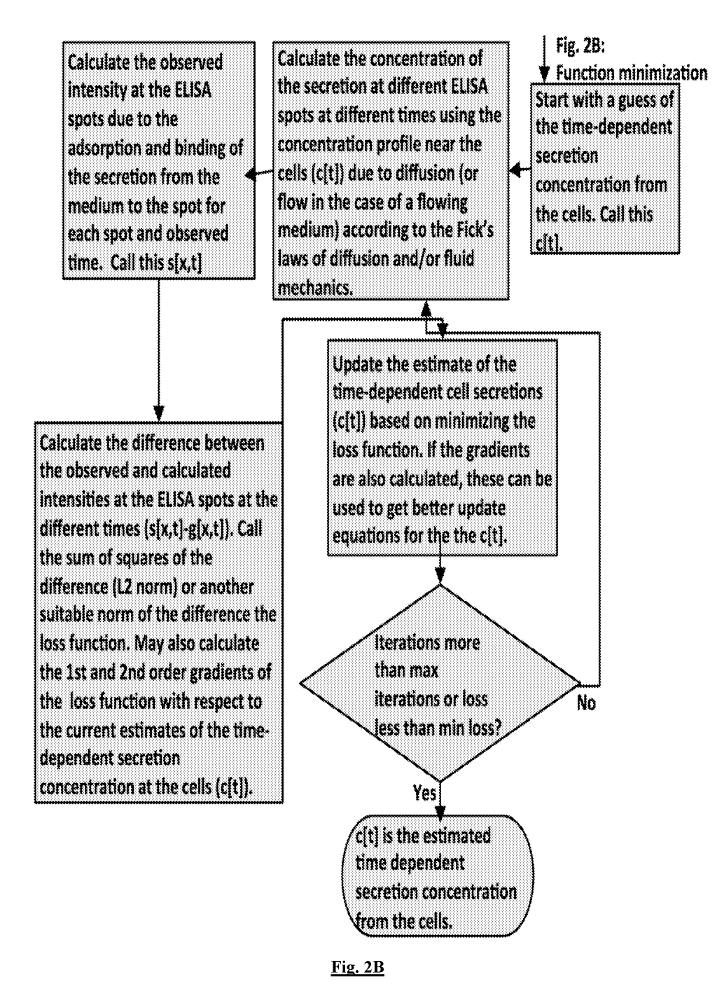

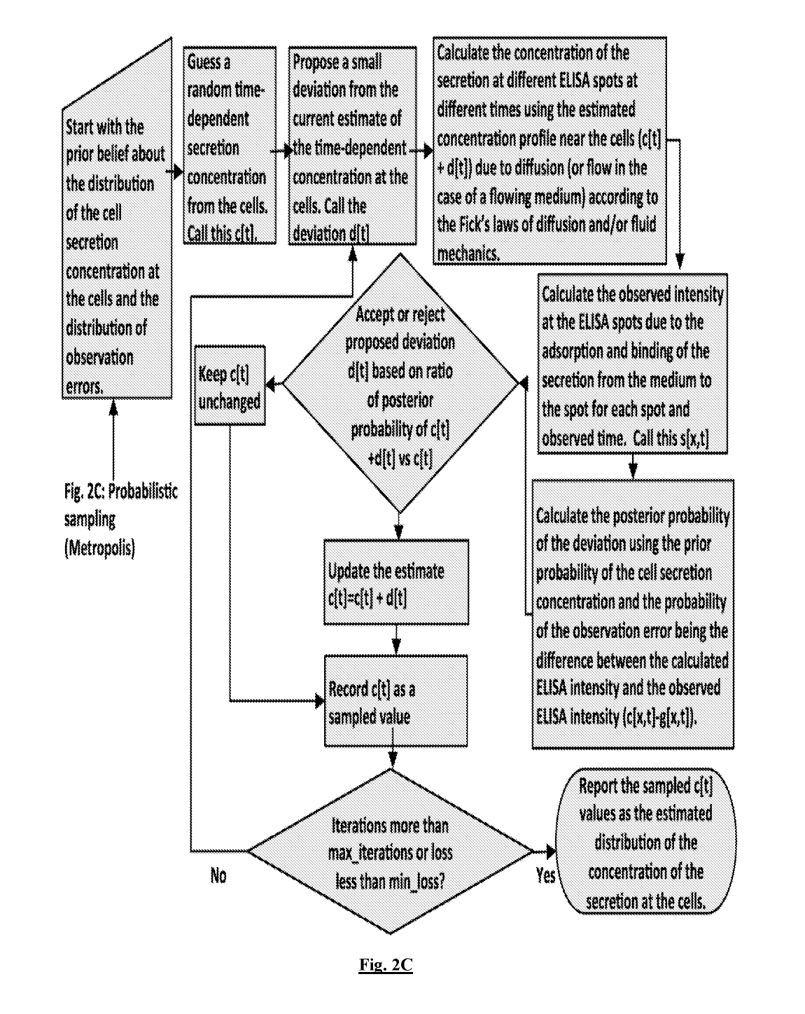

The device of the present invention shown in FIG. 1A comprises an experimental chamber, 10, and an observational chamber, 20. In one embodiment, the experimental chamber and observational chamber are separated by a permeable membrane, 30, such as a plurality of pillars. When liquid is placed in the experimental chamber and the observational chamber is devoid of liquid, a liquid-air interface and distance between the pillars create a surface tension between the two chambers. The surface tension, thus, serves as a barrier to isolate the contents of the experimental chamber from the contents of the observational chamber. Applying a hydrostatic pressure to the experimental chamber that is sufficient to overcome the surface tension generates a laminar flow moving the liquid from the experimental chamber into the observational chamber. In one aspect, the invention includes a device for the temporal high-throughput measurement of one or more molecules secreted by a cell using quantitative enzyme linked immunosorbant assay (qELISA), the device comprising an experimental chamber and an observational chamber, wherein the experimental chamber and the observational chamber are separated by a plurality of pillars, wherein the pillars are selected so that fluidic movement between the pillars is hindered when the observational chamber comprises air and/or is free of liquid.

In another embodiment, the experimental chamber comprises a biological component, such as cells, 50. The biological component is capable of secretion of a molecule or compound that is measured in the observational chamber. In one embodiment, the experimental chamber allows the adhesion of the cell, such as coated with one or more reagents so the cells adhere to a surface of the experimental chamber.

In another embodiment, the observational chamber as shown in FIG. 1A comprises one or more molecule or compound detection locations, 41, such as antibody microspots, or an array of detection locations, 40, i.e., antibody microspots, arranged transversely to the cell-containing experimental chamber. The laminar system of the present qELISA platform allows for the determination of the identity of each detectable molecule or compound, 70, such as an antigen or a captured ligand, as shown in FIG. 1A, the distance to which it has diffused, and the time of secretion. Therefore, it is possible to construct back the temporal profile of secretions by a single snapshot of qELISA at the end of the experiment. If observation is made at multiple time points, spatial information can be used to substantially increase the temporal resolution of the secretion profile for a given molecular species. Since qELISA platform of the present invention is essentially high-throughput in nature, it allows for a high-throughput analysis of the time course of secretions from cells. From a single slide, the qELISA platform of the present invention can derive a very rich information set comprising kinetics of secretions in a high-throughput manner for any adherent/non adherent cell type, cultured under distinct conditions.

In another embodiment, the device shown in FIG. 1A further includes one or more standardization chambers, 60. The standardization chamber is used to calibrate the measurement of the molecule or compound. In one embodiment, the device comprises three standardization chambers. In another embodiment, the one or more standardization chambers is adjacent to the experimental chamber. In still another embodiment, the one or more standardization chambers is adjacent to the observational chamber. In yet another embodiment, the one or more standardization chambers is connected to the observational chamber.

In one embodiment, a liquid, such as culture media, is present in the experimental chamber and a hydrostatic pressure is applied to the experimental chamber. The hydrostatic pressure is sufficient to overcome the surface tension between the experimental chamber and the observation chamber. After applying the hydrostatic pressure, a laminar flow is generated that moves the liquid from the experimental chamber into the observational chamber. The antibody microspots in the experimental chamber detect the identify of specific molecule(s) or compound(s) secreted by the cells and present in the liquid that migrated into the observational chamber and the distance the molecule(s) or compound(s) have diffused to determine the time of secretion.

In one aspect, the invention includes a method of calculating an intensity of a cellular secretion using the device described herein. The method comprises a) contacting cells with the experimental chamber, b) exposing the cells to experimental conditions to induce secretion of the one or more molecules or compounds, c) moving the one or more molecules or compounds from the experimental chamber into the observation chamber, d) binding the one or more molecules or compounds to one or more molecule or compound detection locations in the observational chamber, and e) calculating an intensity of the one or more molecules or compounds.

In another aspect, the invention includes a method of generating a temporal intensity profile of one or more molecules or compounds secreted from a cell. The method comprises a) calculating an estimated intensity of the one or more molecules or compounds at a distinct molecule or compound detection location and time based on diffusion of the one or more molecules or compounds to the detection location (g[x,t]); b) calculating an observed intensity at the detection location due to an adsorption and binding of the one or more molecules or compounds to the detection location at an observed time (s[x,t]); c) calculating a difference between b) and a) (s[x,t]−g[x,t]) to obtain a loss function; d) updating the estimated intensity to minimize the loss function; e) generating the intensity profile for the one or more molecules or compounds at the detection location; and repeating steps a) through e) for a plurality of detection locations, thereby training a function minimization algorithm to generate the temporal intensity profile of the one or more molecules or compounds secreted from a cell.

In yet another aspect, the invention includes a method of generating a temporal concentration profile of one or more molecules or compounds secreted from a cell. The method comprises a) calculating an estimated concentration of the one or more molecules or compounds at a distinct molecule or compound detection location and time based on diffusion of the one or more molecules or compounds to the detection location (c[t]), b) proposing a deviation (d[t]) from the estimated concentration (c[t]+d[t]), c) calculating an observed concentration at the detection location due to an adsorption and binding of the one or more molecules or compounds to the detection location at an observed time (s[x,t]), d) calculating a difference between b) and c) (c[t]+d[t]−s[x,t]) to obtain a posterior probability of the deviation, e) accepting or rejecting the proposed deviation of d[t] based on the ratio of the posterior probability of (d) compared to the estimated concentration a), f) generating the concentration profile for the one or more molecules or compounds at the detection location, and g) repeating steps a) through f) for a plurality of detection locations, thereby training a function minimization algorithm to generate the temporal concentration profile of the one or more molecules or compounds secreted from a cell.

In still another aspect, the invention includes a method of identifying post-translational modification of secreted molecules from a cell in a specific biological condition. The method comprises measuring and determining the kinetics and temporal profiles of the cell's secretory signature in the specific biological condition using the device described herein. The post-translational modification identified can include, but are not limited to, glycosylation, salicylic acid decoration, splicing, polymerization and other post translational modifications.

Methods of detection secretion levels or identifying a particular cell in a subject are also described herein. In one aspect, the invention includes a method of detecting the secretion, level of secretion, temporal intensity profile, and/or temporal concentration profile of the molecule or compound using the device of a molecule by a cell isolated from a subject. The method comprises measuring and determining the kinetics and temporal profiles of the cell's secretory signature using the device described herein.

In another aspect, the invention includes a method of identifying a cell isolated from a subject. The method comprises measuring and/or determining the kinetics and temporal profiles of one or more molecules or compounds using the device of described herein, wherein the profiles identify at least one selected from the group consisting of cell type, cell state, such as cell signaling, cell fate, cell age, and/or cell cycle, and cell response to a biological stimuli.

The present invention also includes methods of treatment. In one aspect, the invention includes a method of treating a disease or disorder in a subject in need thereof, wherein the treatment is cell-free. The method comprises the steps of identifying a first temporal intensity profile and/or temporal concentration profile for one or more molecules or compounds secreted by a cell that is used for treating the disease or disorder, wherein the first profiles comprise one or more biological molecules, identifying a second temporal intensity profile and/or temporal concentration profile for one or more molecules or compounds secreted by various cell types used to treat the same disease or disorder, wherein the second profiles comprise one or more biological molecules, and administering to the subject a therapeutically effective amount of the one or more molecules comprised in either the first or the second profiles, wherein the subject is not administered a therapeutically effective amount of the cell. In one embodiment, cell comprises at least one selected from the group consisting of stem cell that secretes anti-apoptotic factors, stromal cell that secretes multipotency or differentiating factors, immune cell that secretes chemokines that inhibit cancer, immune cell that secretes chemokines that support cancer invasion secreted, and cancer cell that secretes a chemokine that promotes angiogenesis.

In another aspect, the invention includes a method of identifying post-translational modification of secreted molecules from a cell in a specific biological condition, the method comprising measuring and determining the kinetics and temporal profiles of a cell exposed to a specific biological condition. In one embodiment, the modification includes but is not limited to glycosylation, salicylic acid decoration, splicing, polymerization and other post translational modifications.

In another aspect, the invention includes a composition comprising one or more growth factors selected from the group consisting of VEGF, SDF-1α, FGF8, IGF1, insulin, HGF, EGF, IGF1, and SCF, wherein the composition provides cytoprotection and prevents cellular apoptosis when contacted with a cell. In one example, the composition includes IGF1, HGF and SDF-1α. In another aspect, the method is included for treating or preventing a disease or disorder, such as cardiac injury, in a subject in need thereof. The method comprises administering to the subject the composition described herein.

In yet another aspect, the invention includes a composition comprising one or more molecules, wherein the composition preconditions cells with mechanical and hypoxic preconditioning to induce a desired response, such as cell survival, prevention of cell proliferation, cell differentiation, cell multi- or pluri-potency, cell migration, and other cellular phenotypes.

In certain embodiments, the present invention allows for the absolute detection of secretions of cells in an arbitrary biological context, or in response to an arbitrary stimulus, in a high-throughput manner. In other embodiments, high-throughput secretory signatures of a cell are determined in a precise manner in two or more distinct physical environments. In yet other embodiments, high-throughput kinetics of the protein secretions of cells are estimated, creating a unique temporal profile of cell secretions in two or more distinct physical environments.

In certain embodiments, the present invention allows for the identification of a multi-molecular and temporal signature of cells defining their identity, biological state, physiological or pathological context, or response to a stimulus. In other embodiments, the present invention allows for uniquely predicting the temporal responses of known secreted molecules that can be detected by an immunoassay, or to predict modifications in secreted molecules. In yet other embodiment, the present invention allows for measuring absolute secretions of adherent and/or non-adherent cells with precisely defined perturbations and observations. In yet other embodiments, the present invention allows for absolute measurements of cellular secretions in response to other cell secretions in a heterotypic multi-cellular context.

In certain embodiments, chemical modification of a secreted molecule is determined by change in its diffusivity in two or more physical environments. In other embodiments, high-throughput absolute secretion profiles of cells are used to provide unique identifiers to distinct cells, or state of cells either not distinguishable or poorly distinguishable by other methods.

In certain embodiments, the present invention allows for measuring secretions of stem cells and stromal cells that may be responsible for reported amelioration in various injured tissues, as well as secretions of immune cells in response to an insult. In other embodiments, the present invention allows for precisely measuring secretions of stem cells that are known to limit cardiac disrepair, and even provide benefits in the context of other tissue injuries, notably the brain.

In certain embodiments, the present invention relates to a composition comprising one or more secreted factors. In other embodiments, the compositions of the present invention, optionally combined with hypoxic and mechanical preconditioning, significantly enhances cell survival in peroxide-induced injury, ischemia reperfusion, or post transplantation at the site of myocardial infarction. In yet other embodiments, the compositions of the invention have anti-apoptotic effects.

In certain embodiments, high-throughput secretory signatures of a cell are determined in a precise manner in two or more distinct physical environments. In other embodiments, high-throughput kinetics of the protein secretions of cells are estimated, creating a unique temporal profile of cell secretions in two or more distinct physical environments. In yet other embodiments, chemical modification of a secreted molecule is determined by change in its diffusivity in two or more physical environments. In yet other embodiments, high-throughput absolute secretion profiles of cells are used to provide unique identifiers to distinct cells, or state of cells either not distinguishable or poorly distinguishable by other methods.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1: qELISA: Microfluidics-Based ELISA Platform to Quantitatively Detect Cell Secretion

To investigate cell secretions of adherent cells in a high-throughput manner, and also obtain an estimate of their secretory dynamics, a device combining the strengths of protein microprinting and microfluidics was created. Protein microprinting allows for high-throughput ELISA-based measurements, while microfluidics facilitates movement of secreted molecules.

Since movement of molecules is restricted to diffusion in a microfluidic device, the distance traveled by a molecule can be decoded for its time stamp. Taking advantage of this property, a qELISA platform was designed allowing for high-throughput detections of cellular secretions, measurements after a variety of biological stimulations, and also estimations of kinetics of cellular secretions (FIG. 1A). Using this device, a secretory signature can be determined in the cellular response to an arbitrary biochemical stimulus (FIG. 1B). Many stem cell types rescue host tissues in response to injury, possibly through their paracrine signaling. Determining absolute concentrations of distinct molecules in cellular secretions allows for the preparation of compositions that mimic the cellular secretory profiles. Using the device can therefore mimic the paracrine signaling provided by BMSCs that prevent myocardial death, obviating the need of cells and creating a cell-less therapy (FIG. 1C).

In certain embodiments, the qELISA platform comprises an experimental and an observational chamber, and three standardization chambers. Cells are seeded in the experimental chamber, and after adhesion they can be subjected to any biological stimulus. The observational chamber comprises rows of antibodies, each row consisting of potentially an antibody recognizing a distinct protein in the secretions from the cells (FIG. 1D). Cellular phenotypes, including their secretions, are dependent on the biochemical cues presented to them by their microenvironment, or neighboring cells.

Precise measurement of the phenotype as a response to a biological stimulus is difficult. To overcome this challenge, the experimental chamber and the observational chamber containing the qELISA spots were fluidically separated with a hexagonal pillar array designed to prevent fluidic movement across under normal pressures due to surface tension. This allows the user to prevent cell media from contacting the detection area until an arbitrary desired start time. This function is performed by placing cells and cell media in the left chamber, while leaving the detection area filled with air. The resultant air-liquid interface between the pillars keeps the liquid confined to the cell area (FIG. 1E), until the arbitrary time in which the user floods the right chamber and initiates the use of the detection system. Cell secretion can therefore be measured in any arbitrary time interval, and timed to match changes in culture conditions. ComSol simulation (FIG. 1F) demonstrated that due to relatively small pillar size and effects of diffusion, the pillars do not unduly disrupt the progression of the ligands.

The capability of the qELISA chip was compared with a flow cytometry-based method to detect protein secretions. BMSCs, subjected to normoxia or hypoxia for 12 hours were treated with brefeldin-A to block secretion for the duration of the experiment, and fixed with paraformaldehyde to freeze the secretory vesicles inside the cells. Cells were permeabilized, stained with specific antibodies against HGF, VEGF, IGF-1, DKK1, SDF-1α, and IL6 and analyzed using flow cytometry. Secretions of cells subjected to similar experimental conditions were analyzed using qELISA.

A high correlation in the ratio of the amount of secretions from BMSCs subjected to normoxia and hypoxia was found when measured by flow cytometry or qELISA (FIG. 1G). These data suggest that the qELISA platform can reliably detect cellular secretions, while offering the advantage of high-throughput analysis, and measurements of secretion kinetics. Since qELISA can also measure absolute amounts of protein secretions with little inter-experimental variability, the effect of hypoxia on BMSC secretions was also tested (FIG. 1H). In normal culture conditions, BMSCs secreted an appreciable amount of molecular species that have been previously reported to be cytoprotective, including HGF, VEGF, IGF1 and SDF-1α, in addition to DKK1, and IL6 (FIG. 1H). Interestingly, when BMSCs were cultured in hypoxia, increased secretions of DKK1 were detected, while HGF, and IGF1 secretions were reduced (FIG. 1H). Surprisingly BMSCs did not increase VEGF secretion in response to hypoxia, though this behavior was also observed in these cells in other studies. Without wishing to be limited by any theory, BMSCs might have a very limited basal capacity to translate and secrete VEGF.

### Predicting Temporal Profiles of Cell Secretions.

Secretion is one of the most common and effective ways of cell-cell communication. Tightly controlled dosage, as well as temporal regulation of secretion of each molecular species is necessary to maintain homeostasis, and to affect an appropriate response to a biological stimulus. Cellular secretions can be in form of a sustained release of a molecular species, or in form of an impulse, an oscillatory wave, or exhibiting a more complex kinetic profile. Owing to the difficulty of precisely measuring cellular secretions, temporal dynamics of most cellular secretions are not studied at all, or are poorly understood. Estimating the dynamics of cellular secretions is necessary to understand intercellular communication. BMSCs are known to provide beneficial effect in many injured tissues with possibly distinct mechanisms of cytoprotection. It is thus possible that the differential effects may be obtained by not merely distinct multidimensional molecular signatures, but also by distinct temporal dynamics of secretions.

A unique advantage offered by microfluidics is that fluid flow is laminar in nature, allowing for diffusion to be the only way for spatial movement of molecules. Diffusion displacement of a molecule in the absence of flow encodes its temporal history. Therefore spatial information can be potentially translated into temporal/historical information. The qELISA platform of the present invention has antibody microspots arranged transversely to the cell containing experimental chamber. In the laminar system of the present invention, for each detectable antigen, the distance to which it has diffused can determine time of secretion. Therefore, it is possible to construct back the temporal profile of secretions by a single snapshot of qELISA at the end of the experiment. Indeed, if observation is made at multiple time points, spatial information can be used to substantially increase the temporal resolution of the secretion profile for a given molecular species. Since qELISA platform is essentially high-throughput in nature, it allows for a high-throughput analysis of the time course of secretions from cells. From a single slide, qELISA platform can therefore predict a very rich information set consisting of kinetics of secretions in a high-throughput manner for any adherent/non adherent cell type, cultured under distinct conditions.

To predict the intensity profiles that can be obtained from commonly occurring simple secretion profiles, a computational model was created based on the hypothesis that for each microspot the intensity (or amount of the captured antigen) is the integral over time of the amount of antigen that has diffused at that distance. This was made under the assumptions that the antibody-antigen binding is essentially irreversible, and that there is no saturation of the antibodies tethered at a given microspot. The ODE-based forward mathematical model was able to predict qELISA intensity profiles in response to simple secretory profiles. Further, it was attempted to construct back the secretion kinetics from the qELISA intensity signatures.

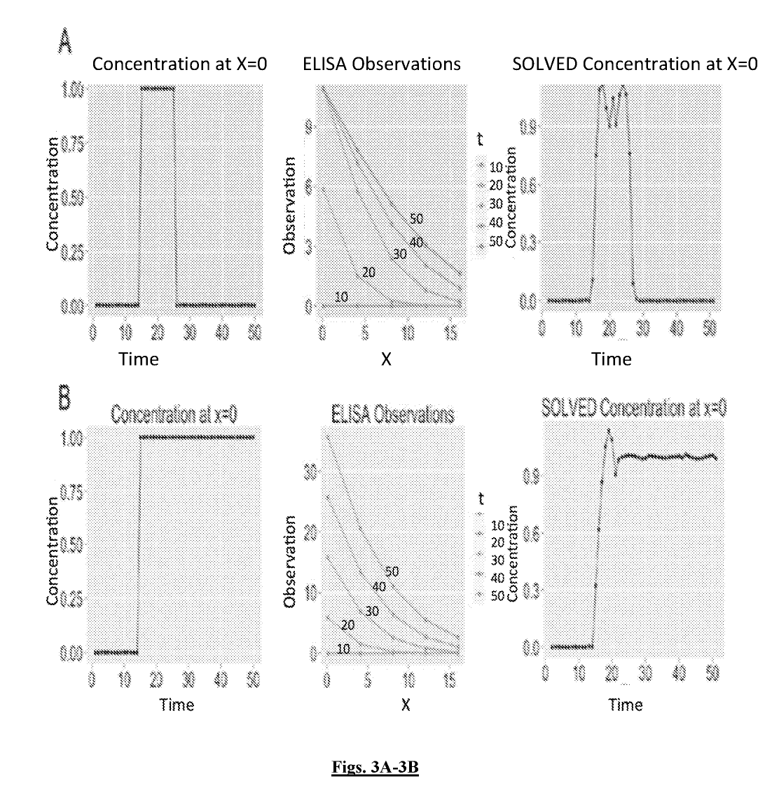

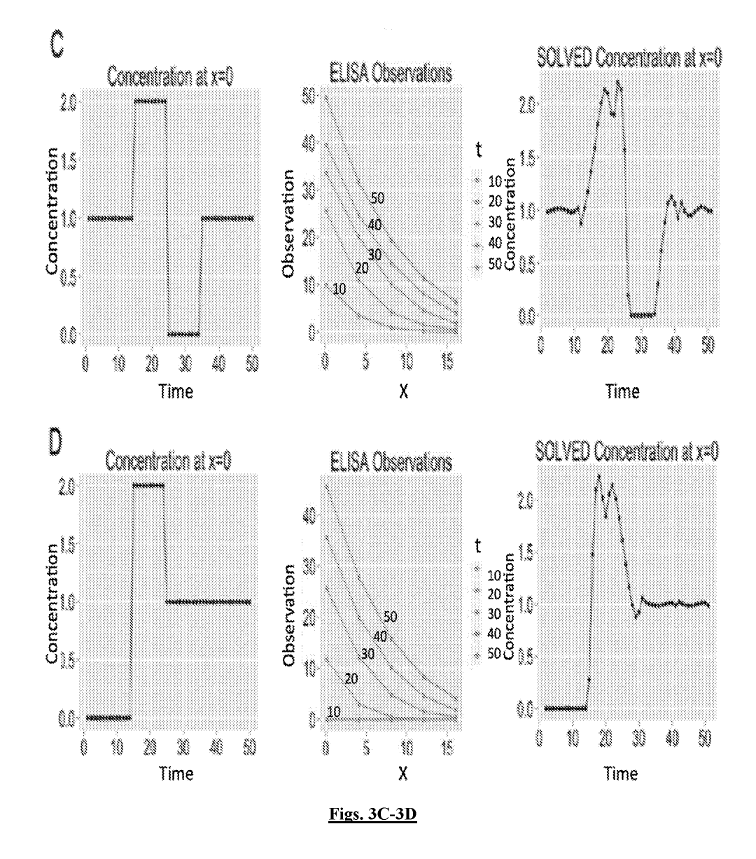

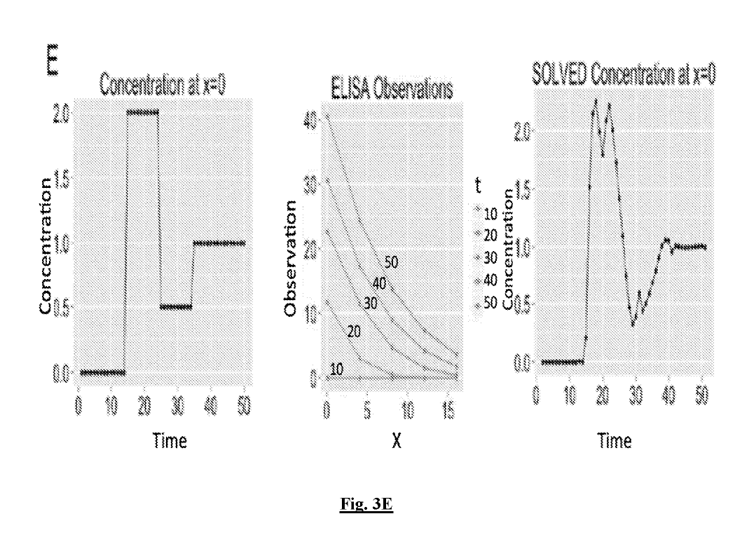

Simulations were performed on the model of the present invention with commonly occurring examples of secretory kinetics in cells. In particular, an estimation was performed for the intensity signatures created from cell signature in the qELISA platform of the present invention, when the secretion is pulsatile (FIG. 3A), a step function (FIG. 3B), a wave (FIG. 3C), an impulse followed by a plateaued kinetic (FIG. 3D), an impulse followed by an overshot dip followed by a plateaued kinetic (FIG. 3E). The forward model of the present invention predicted the intensity signatures in qELISA platform observed at distinct time points, as well as concentration of the secreted molecular species in space.

In response to an impulse secretion (FIG. 3A), the concentration of the molecule in the observation chamber showed a widening impulse function as the diffusion distance is proportional to the square root of time (FIG. 3A). A pulse wave results in a characteristic intensity profiles in the qELISA observation chamber, with increasing intensities further away from the experimental chamber over time. Using the probabilistic reverse model of the present invention, the kinetic profile of secretion was recreated from the intensity signatures. The most probable estimate of the derived kinetics closely matched the input function in all tested cases.

### Cell Formulate a Context Specific Secretory Signature.

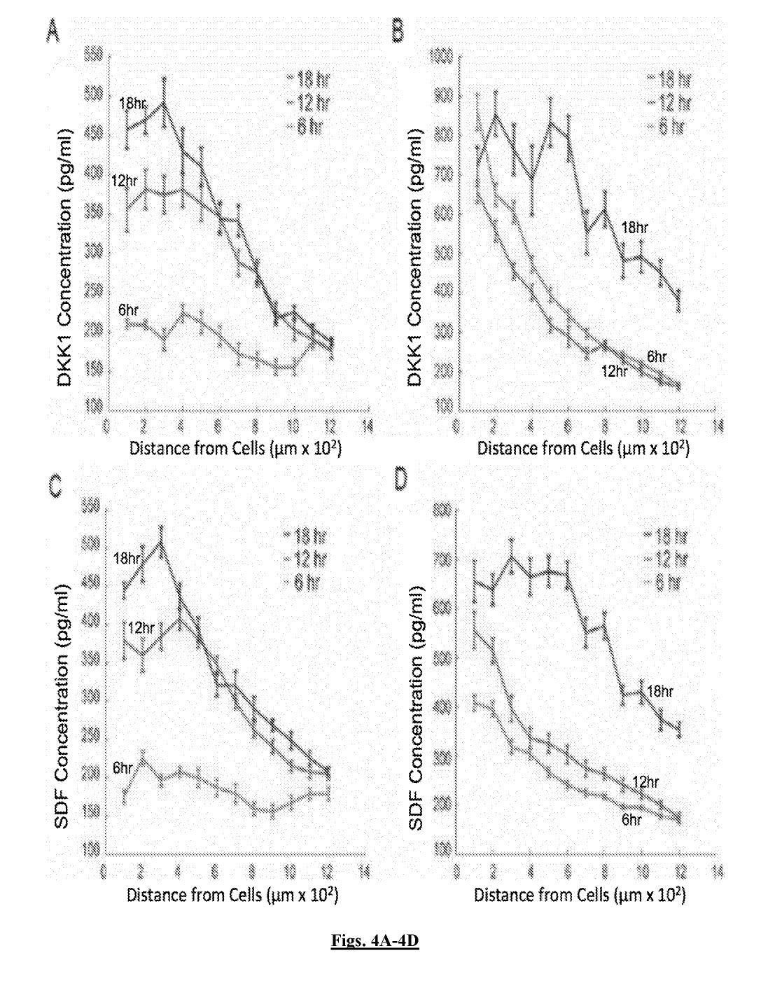

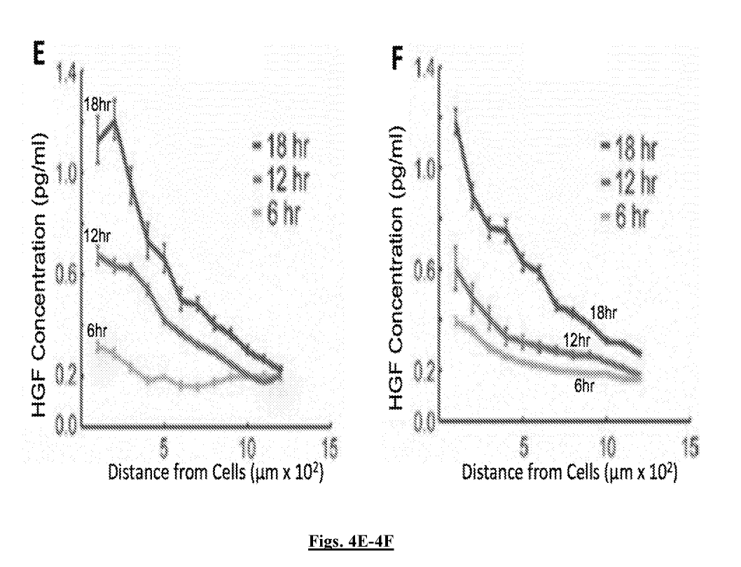

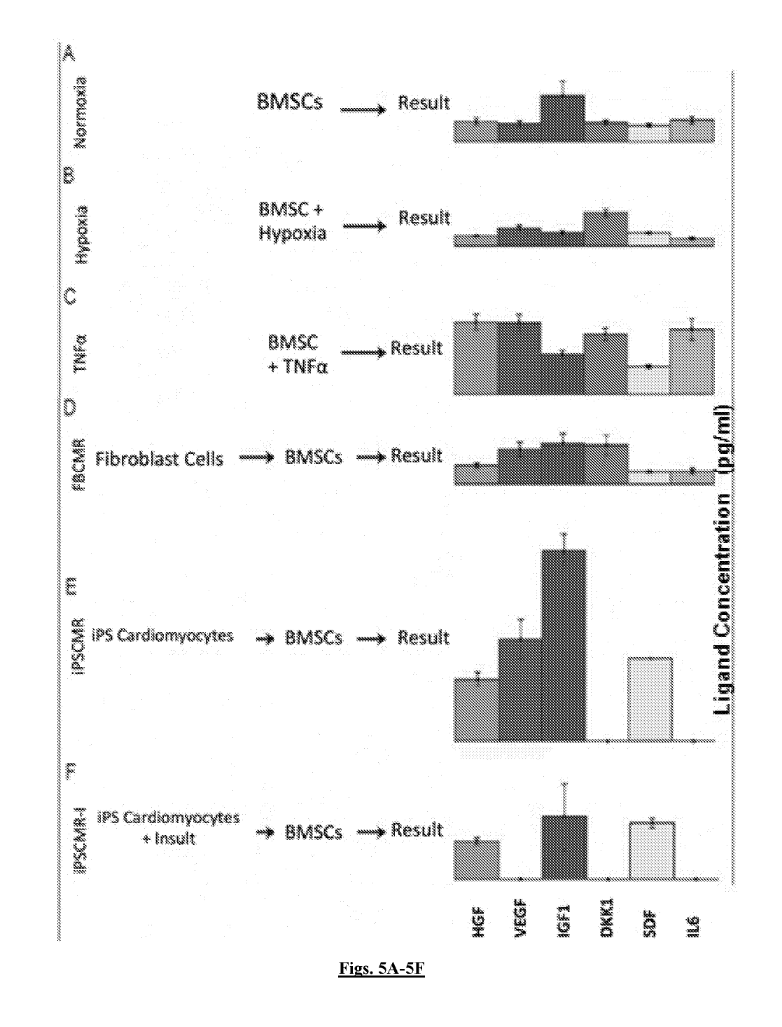

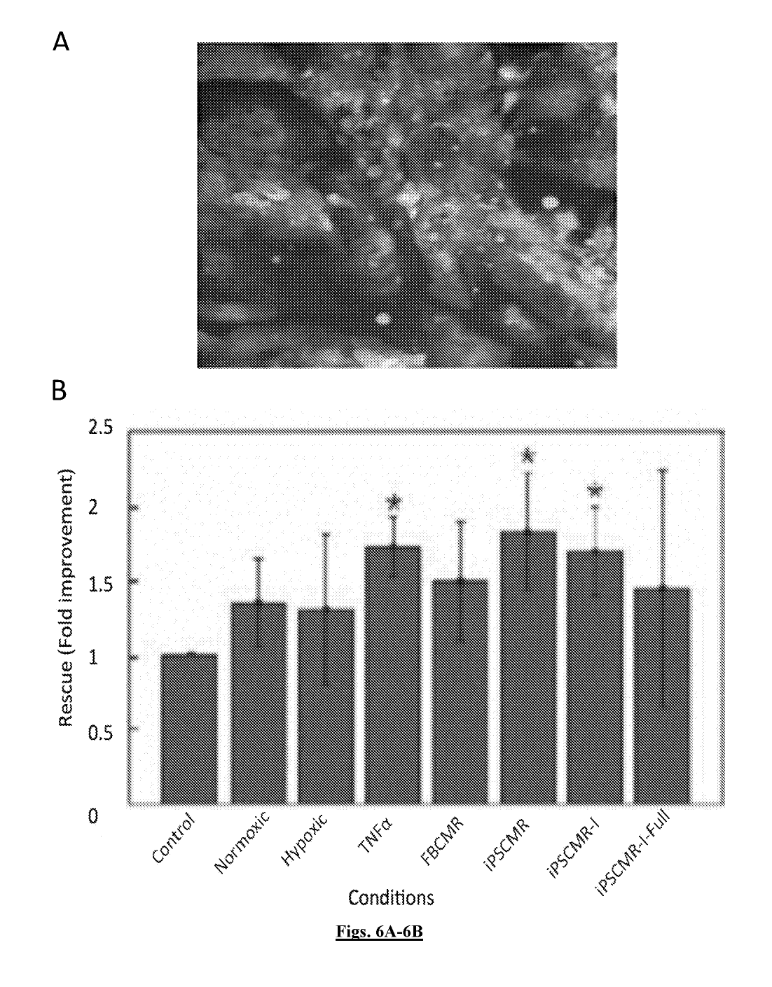

BMSCs secretions were distinct in hypoxia as compared to normoxia. Since in a device with diffusion being the only method for molecular movement, temporal information can be derived from spatial information, the amounts of molecules detected were carefully analyzed as a function of distance from the experimental chamber. The intensity of microspots coated with antibody were measured against a selection of secretory molecules at increasing distances from the experimental chamber 6 hours, 12 hours, and 18 hours after the start of the observation. BMSCs secretions under hypoxia differ not only in absolute amounts for DKK1 (FIG. 4A-B), SDF-1α (FIG. 4C-D), and HGF (FIG. 4E-F), but the rate of secretions were also distinct. Since absolute amount of a molecular species detected at a given distance is a proportional function of the integral over time of the rate of its secretion, the increased amounts of DKK1 secretion in response to hypoxia was sustained, while in normoxia BMSCs secrete DKK1 at a non-constant rate. Similar changes in the kinetics of secretions were observed for other molecular species analyzed. These data highlight the fact that cell secretions are altered in response to a biological stimulus, and it is inaccurate to consider secretions from a cell type without a biological context. Further, not only cell secretion for each molecular species can be a function of the biological stimulus, but also the kinetics of its secretion. The algorithm of the present invention was used to predict the temporal profiles of HGF secretion by BMSCs in response to hypoxia from their observed qELISA intensity profiles (FIG. 5). Interestingly, hypoxia had not only an effect on the total secretion of HGF, but also drastically changed the secretion kinetics, with potentially very significant effect on the target cells of BMSCs. qELISA platform allows an extremely rich data generation of secretory profiles of cells in throughput, biological context, as well as in its temporal nature.

### Injured Cardiac Cells Induce BMSCs to Secrete Anti-Apoptotic Factors.

A wide variety of stem cells including BMSCs limit damage to injury without direct differentiation. To test the hypothesis that the reported benefit offered by BMSCs is due to paracrine effects, the qELISA platform was used to measure their secretions in the presence of factors present in the infarct. Though BMSCs do secrete many known cytoprotective factors (FIG. 1H), however it is possible that their secretions are more attuned to their reported function of cytoprotection when they are present at the site of injury. Therefore BMSCs were treated with hypoxia, and an inflammatory cytokine TNFα known to be produced at the site of infarct (FIG. 5A-C). QELISA analysis indicated that while compared to normoxia (FIG. 5A) hypoxia increased secretion of DKK1 and decreased secretion of HGF, IGF-1, SDF-1α and IL-6 (FIG. 5B), TNF-α stimulation increased the secretions of all the molecular species investigated (FIG. 5C).

Hypoxia and TNFα, both present at the site of infarct have dissimilar effect on BMSC secretion, indicating that the response to a complex biological stimulus cannot easily be elicited by stimulation with a single constituent factor of the stimulus. Since it is difficult to individually isolate each biochemical factor present in the complex environment of MI, a model for MI was created using human pluripotent stem cells derived cardiomyocytes (iPSCMR). To investigate the secretions of BMSCs in response to MI, BMSCs were conditioned with medium collected from iPSCMRs treated with Imatinib, an oxidative stress inducer. In addition, BMSC secretion was also analyzed when conditioned with medium collected from uninsulted iPSCMRs, and uninsulted cardiac fibroblasts (FBCMR).

QELISA analysis of BMSCs with conditioned medium from FBCMR (FIG. 5C) exhibited a similar profile to control untreated BMSCs (FIG. 5A). However, the secretory signature of BMSCs in response to conditioned medium from iPSCMR changed drastically, showing no detectable DKK1 levels, while significant increase in HGF, VEGF, IGF-1, IL-6 secretions (FIG. 5E). Surprisingly, BMSCs treated with conditioned medium from insulted iPSMR exhibited a dramatically distinct secretory signature with no detectable VEGF, DKK1 and IL-6 secretions, and selectively secreting HGF, IGF-1 and SDF-1α (FIG. 5F). These results were also confirmed with flow cytometry.

Since secretions from BMSCs is known to be cytoprotective at the site of MI, in the limited selection of known pro-survival secretory molecules probed this specific secretory signature constitutes the cytoprotective cocktail that prevents redox-induced cell death.

### Recreated Cytoprotective Cocktail Prevents Cardiac Cell Death.

BMSCs are cytoprotective at the site of cardiac infarct in vivo.

First, BMSC secretions were confirmed to be indeed cardioprotective in response to reperfusion injury, the most common reason for cell death in an MI. iPSCMR were cultured in a monolayer with beating cardiomyocytes, and measured the extent of apoptosis after treatment with 100 μM H₂O₂ for 30 minutes in the presence of BMSC conditioned medium. Calcein-AM staining revealed a significantly high cell rescue in the presence of BMSC conditioned medium, as compared to controls (FIG. 6A). It was further tested whether conditioned medium from BMSC pretreated with individual factors present in MI resulted in increased cell rescue. Indeed, compared to control (no presence of conditioned medium), conditioned medium from BMSCs treated with hypoxia significantly rescued cardiac cell death, while conditioned medium from BMSCs treated with TNFα resulted in even higher cell rescue.

The rate of cardiac rescue was measured in the presence of conditioned medium from BMSCs treated with medium from cultures of FBCMR and iPSCMR. While conditioned medium from BMSCs treated with medium from FBCMR cultures did not result in any further increase in cell rescue, those from iPSCMR treated BMSCs increased cell rescue significantly higher. Finally, it was tested whether conditioned medium from BMSCs treated with medium from iPSCMR cultures that were insulted with Imanitib (mimicking ischemia reperfusion insult) had a higher capability of rescuing redox stressed cardiac cells.

The effect of these factors were tested on cardiac function in a mouse model of MI. Secretion containing medium collected from BMSCs after treatment for 6 hours with iPSCMR or Imanitib-treated iPSCMR conditioned medium were injected at the site of infarct 2 hours after ligation. Medium from BMSCs that were untreated was used as control. Echocardiography showed that secretions from BMSCs that were treated with conditioned medium from iPSCMR (Iminitib treated) improved cardiac function substantially as compared to the control (t-test, p-value 0.0001, FIG. 6B).

These data suggest that while BMSCs naturally secrete anti apoptotic factors, they may not be sufficient to prevent peroxide-induced apoptosis of cardiomyocytes. Instead, secretions from BMSCs rescues cardiac cells from peroxide-induced apoptosis in a biochemical context of an infarct. It follows that BMSCs alter their secretory profile in response to the inflammatory, and oxidatively stressed environment in the infarct, to prevent further cell death.

### Identification of a Universal Cytoprotective Cocktail to Limit Cardiac Disrepair.

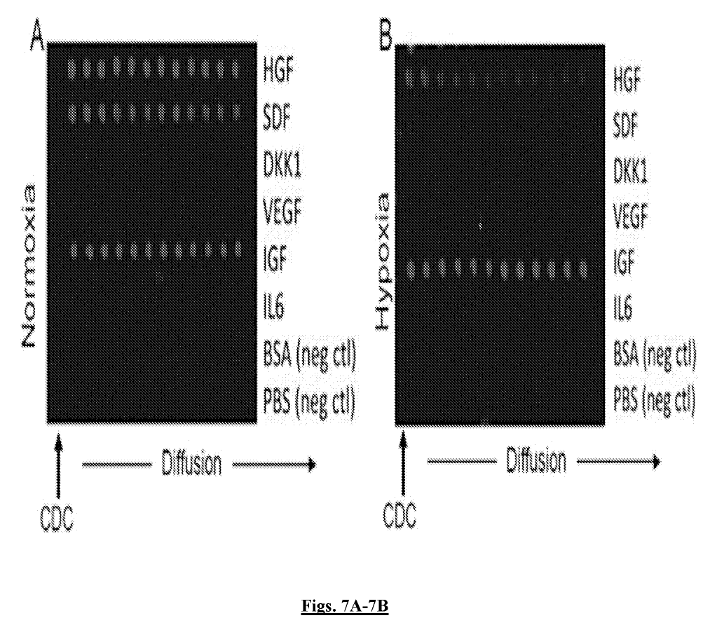

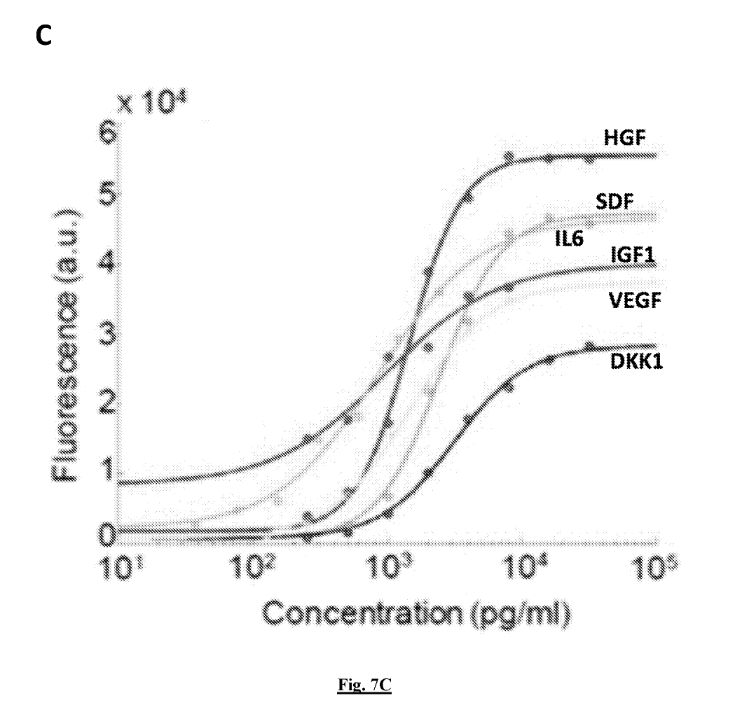

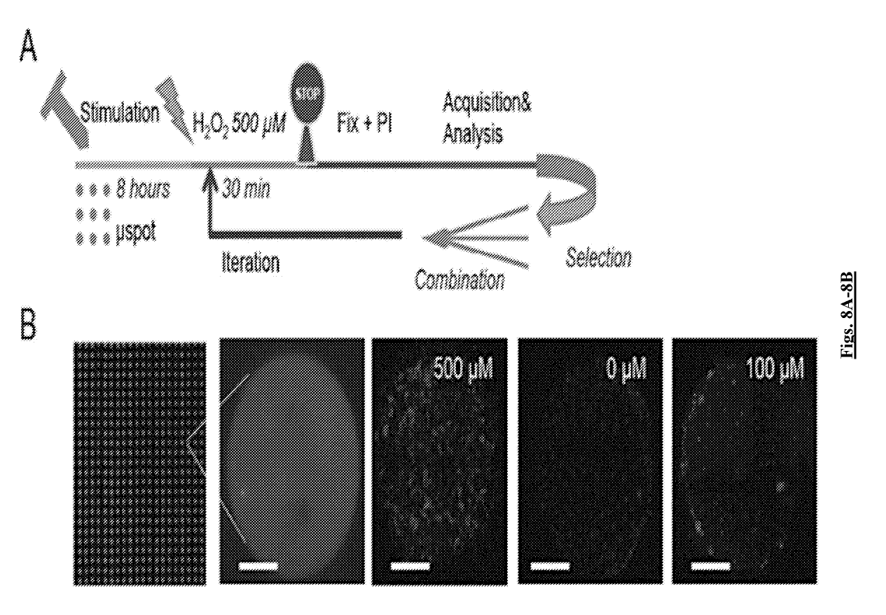

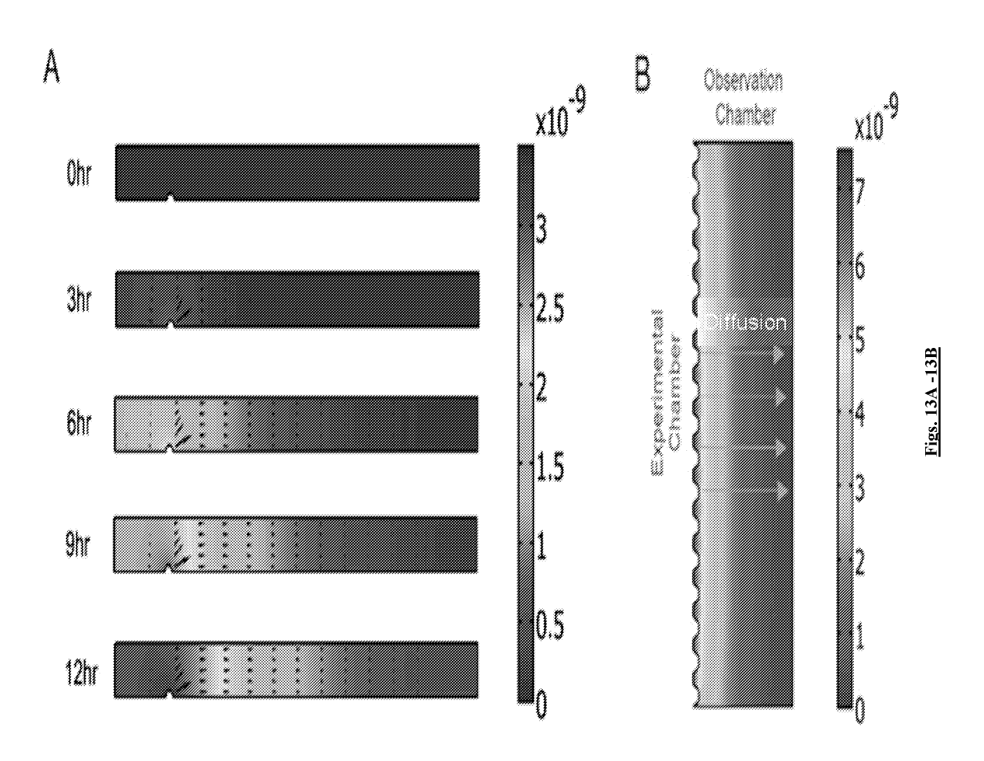

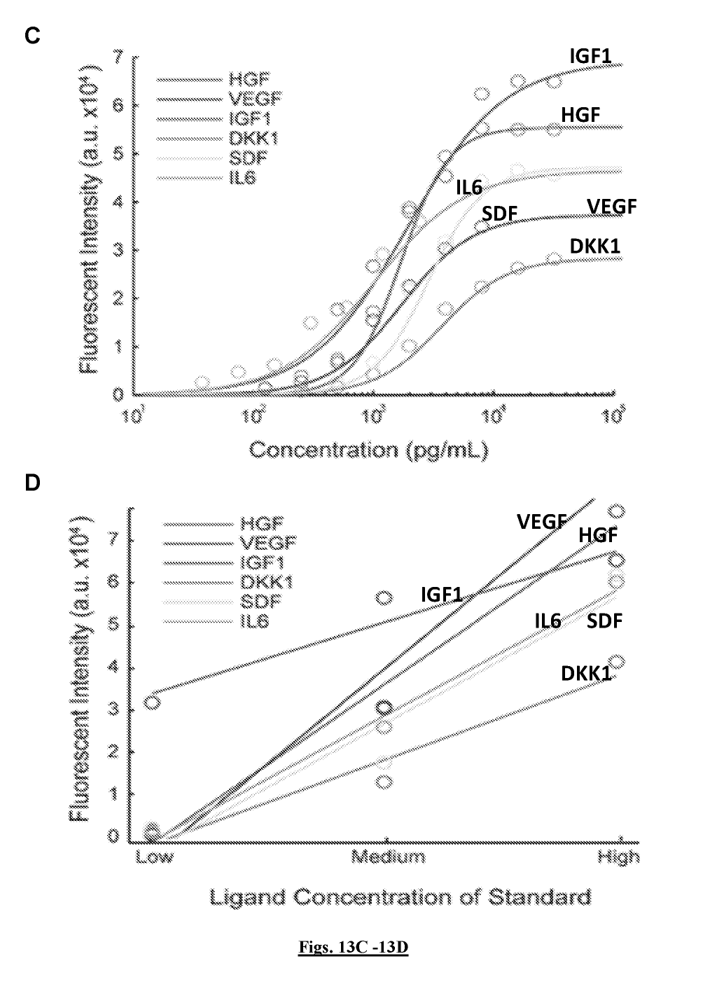

CDCs and BMSCs limit damage when transplanted at the site of myocardial infarction. Using the MicroELISA chip it was attempted to screen factors that these cells secrete under normoxia, and under hypoxia mimicking the oxygen tension in the infarct. In a limited test, 6 ligands previously reported to be present in the secretome of these cells were tested. The fluorescent intensities observed to concentration of ligands, and compared the effect after 18 hours of cell culture in hypoxia, or in normoxia. CDCs were found to secrete IGF1, HGF, and SDF-1α in large amounts in normoxia (FIG. 7A), though secretion of SDF-1α was subdued when cells were cultured in hypoxia (FIG. 7B). BMSCs also secreted HGF, IGF1, IL6, and SDF-1α in appreciable amounts under normoxia (FIG. 5A), however secretion of HGF, IGF1 and IL6 were downregulated, while DKK1, a regulator of Wnt pathway was upregulated when cells were cultured in hypoxia (FIG. 5B). For both CDCs, and BMSCs, standardization curves allowed quantitative determination of the absolute amount of secretion for each of the species measured (FIG. 7C, FIG. 13C, and FIG. 13D).

### Iterative Determination of Minimal Component Cytoprotective Cocktail.

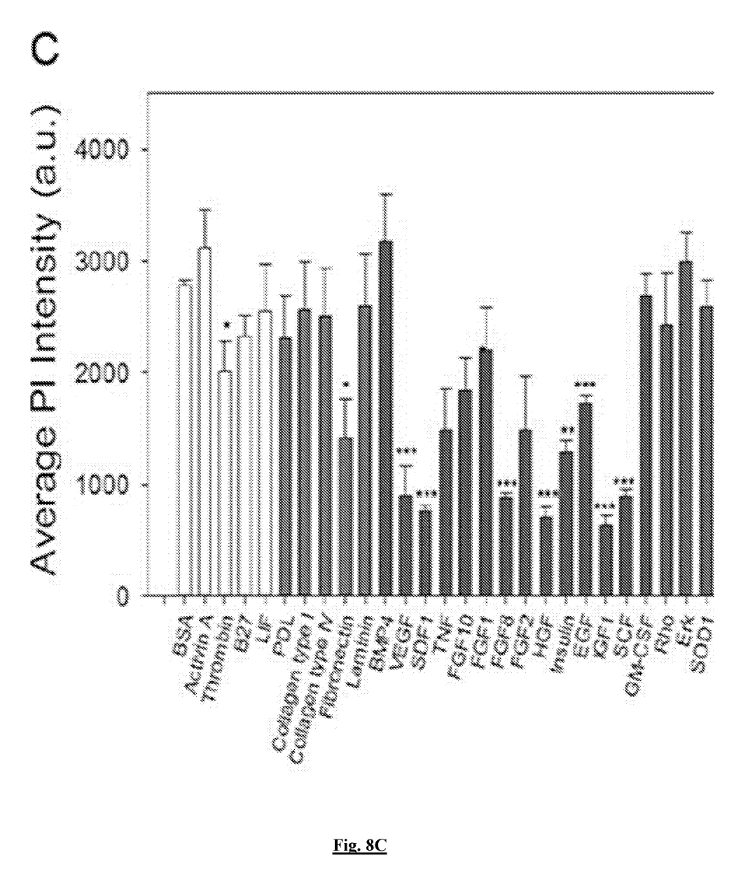

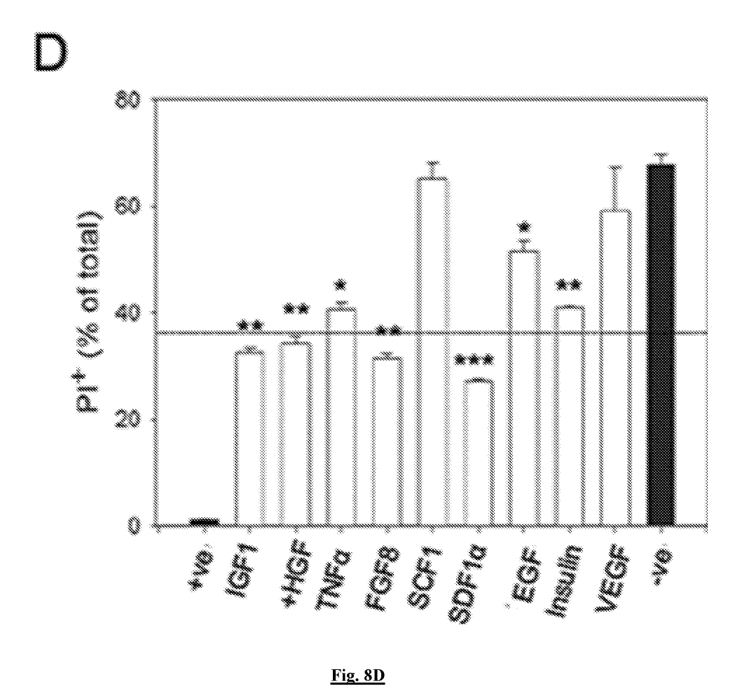

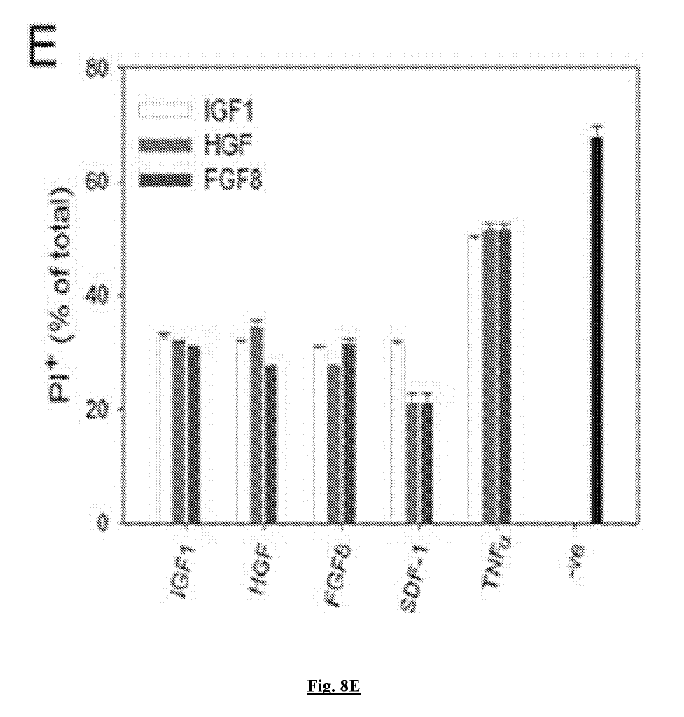

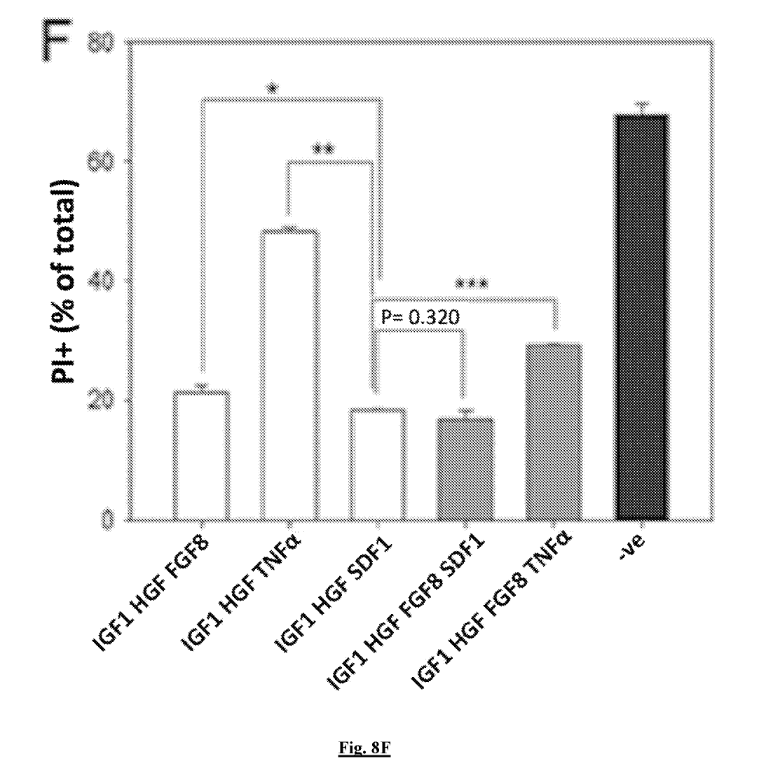

Since peroxide production during ischemia reperfusion is the leading cause of cell death upon cell transplantation at the site of cardiac infarct, the cytoprotective effect of cell preconditioning was assessed by a variety of factors in an in vitro setting. Towards this, an in vitro peroxide-induced cytotoxic assay was set up, consisting of culturing CDCs in polystyrene surfaces and treating with 500 μM H₂O₂ for 30 minutes. Propidium iodide (PI) staining revealed that peroxide treatment was sufficient to kill >90% of cells. This cytotoxic assay was used to assess the cytoprotective effect of various biochemical agents singly, and if the cytoprotective effect was very significant (p<0.001) then the biochemical agents were combined in a combinatorial fashion, and iteratively tested for a combined cytoprotective effect. The process was iteratively repeated till further combinations failed to provide any further significant improvement in cytoprotection, in order to create a minimal-constituent optimal cytoprotective cocktail to prevent peroxide-induced apoptosis (FIG. 8A).

Protein microprinting technology was used to create spots with diameter 100 μm with various potential cytoprotective factors in a range of concentration. The proteins spotted were mixed with fluorescein-conjugated gelatin to ensure CDC adhesion. CDCs were cultured on these microspots for 12 hours and subjected to the in vitro peroxide assay, stained and analyzed for the proportion of PI(+) cells in the total cell population (FIG. 8B). Stained cells were imaged using an epifluorescence microscope equipped with a robotized stage controlled by a customized MATLAB coded driver, and analyzed with a MATLAB coded custom cell counter (FIG. 3B). Image analysis revealed that average PI intensities were significantly decreased in CDCs cultured in spots coated with Thrombin, Fibronectin, VEGF, SDF-1α, FGF8, HGF, Insulin, EGF, IGF1, and SCF while the decrease in average PI intensities were not significant for other biochemical factors when compared to BSA control (FIG. 8C). In particular, VEGF, SDF-1α, FGF8, IGF1, Insulin, EGF, IGF1, and SCF preconditioning resulted in a very significant decrease in average PI intensities in CDCs, and these factors were chosen for further iterative combinations (FIG. 8C). CDCs were cultured in the presence of the above factors for 12 hrs, and subjected to peroxide assay. TNFα was chosen as one of the additional biochemical factors as a control.

IGF1, HGF, FGF8, and SDF-1α were combined in pairs and CDCs were preconditioned for 12 hours with the paired combination (FIG. 8D). TNFα was also used in paired combination with other factors as control. A combination containing SDF-1α was found to significantly reduce CDC toxicity upon application of peroxide, resulting in prevention of cell death over 3 times vs the control (FIG. 8E). Biochemical factors were iteratively combined in groups of 3, and 4 and subjected to peroxide assay. A combination consisting of IGF1, HGF, SDF-1α was found to very significantly reduce peroxide-induced cytotoxicity (FIG. 8F). Further grouping of cytoprotective factors identified in previous iterations failed to further improve cytoprotection significantly, suggesting that the iterative method of combination of cytoprotective factors had arrived at a minimal-constituent optimal biochemical cytoprotective cocktail consisting of 3 biochemical growth factors (FIG. 8F).

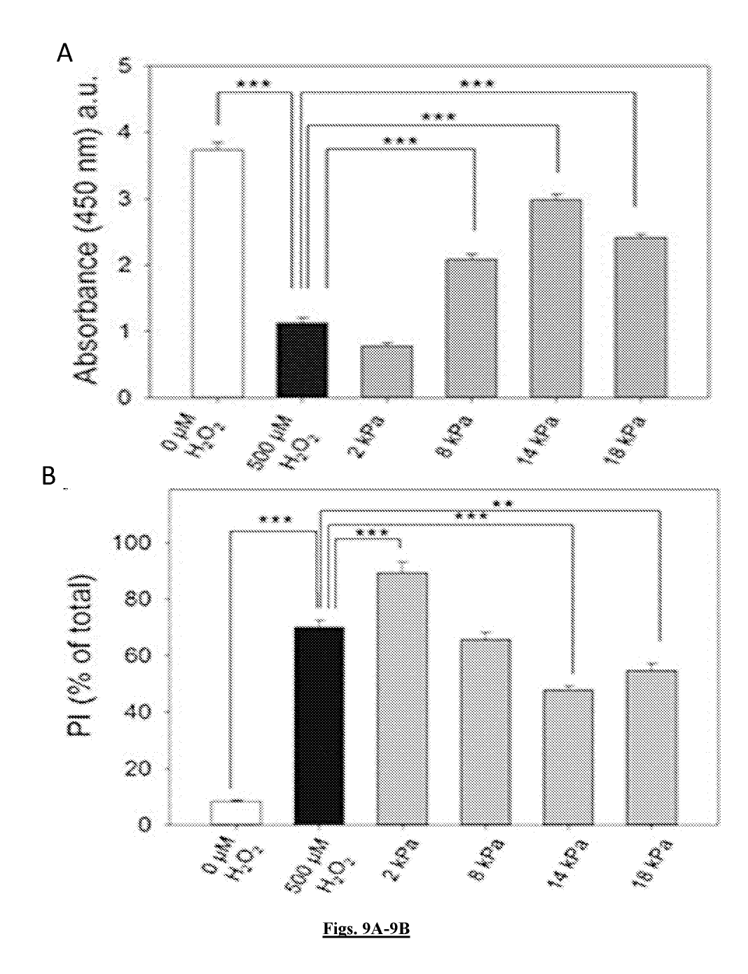

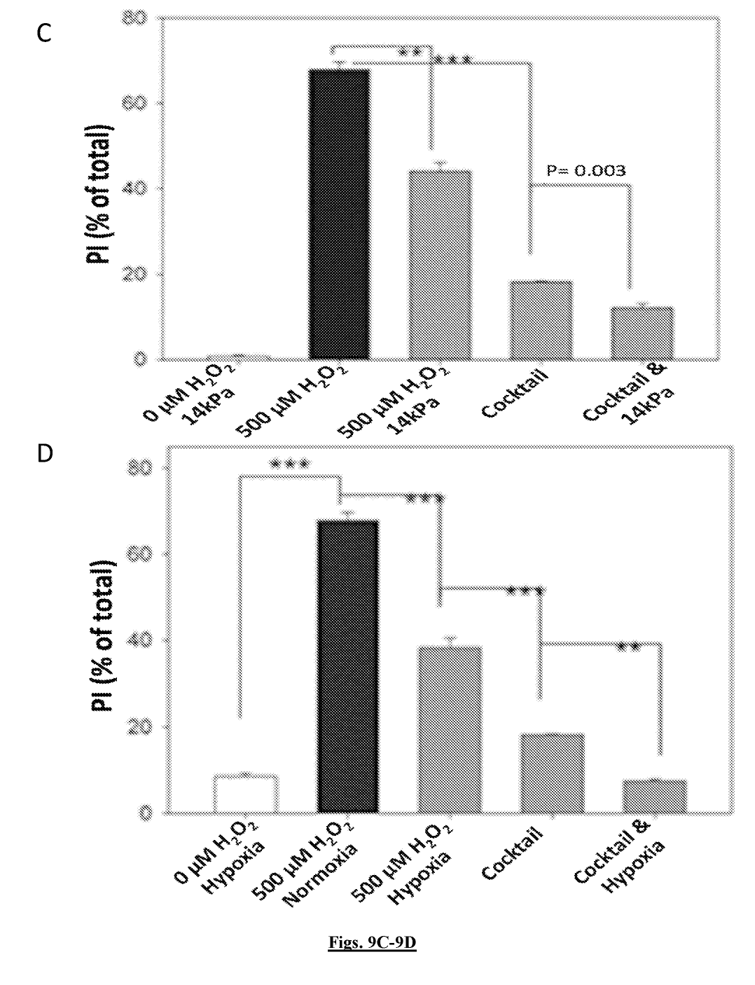

Having identified an optimal biochemical cocktail to prevent cell death in the presence of peroxide, one of the chief cytotoxic agent present during ischemia reperfusion. CDCs have exhibited distinct rates of proliferation in substrata of distinct rigidities. Thus, it was questioned whether substratum rigidities could also influence cellular protection in the presence of external apoptotic stimuli, since adhesion signaling has been widely reported to have an important role in cell survival. Using WST-8 assay to assess cell numbers, CDC survival was estimated when cultured for 3 days on substrata of rigidity mimicking various tissues upon application of peroxide assay. WST-8 assay indicated maximum cell survival on substratum rigidity mimicking the native myocardium (14 kPa), while a soft substratum (2 kPa) resulted in even further increase in cellular apoptosis than the control where cells were cultured on polystyrene surface (FIG. 9A). Flow cytometry revealed a similar trend indicating that myocardium mimicking rigidity substratum significantly enhanced cell survival post peroxide assay (FIG. 9B).

It was also inquired whether culturing CDCs on substratum mimicking myocardium could further enhance cell survival when used in combination with the optimal biochemical cocktail. CDCs were cultured for 3 days on substratum with rigidity of 14 kPa, and on control surface (polystyrene) and preconditioned with the cocktail for 12 hours prior to being subjected by peroxide assay. Flow cytometry revealed a further significant reduction in cell death when MRS and biochemical cocktail were combined, resulting in reduction of cell death to >4 times vs. the control when no preconditioning was provided to the cells (FIG. 9C).

Orthogonal methods by which cells could be protected against peroxide were hypothesized to induce apoptosis. Hhypoxic preconditioning can prevent superoxide-induced cell death in vitro, and in ischemia reperfusion-induced apoptosis in vivo. CDCs were preconditioned for 12 hours in 1% 02, and immediately subjected to peroxide assay (FIG. 9D). Hypoxia itself was found to significantly reduced peroxide-induced cell death vs. the control, though less than the optimal biochemical cocktail preconditioning. Hypoxic preconditioning was further questioned, when combined with optimal biochemical preconditioning could further enhance cell protection against peroxide-induced cell death. Preconditioning CDCs with biochemical cocktail combined with culturing them in 1% O₂ further significantly enhanced cell protection against peroxide-induced cell death (FIG. 9D).

Growth factors typically signal to the cells as soluble biochemical factors, or as tethered entities to the extracellular matrix. In contrast hypoxia and mechanical signals are perceived by cells in distinct ways, though possibly feeding into similar signal transduction subnetworks. Therefore, it was surmised that if the orthogonal cytoprotective agents are combined and presented simultaneously to CDCs, it may result in even further reduction in peroxide-induced apoptosis. Combining substratum rigidity mimicking myocardium, optimal biochemical cocktail, and hypoxic preconditioning together to create a comprehensive cytoprotective cocktail further significantly reduced cell death in the in vitro peroxide assay (FIG. 9E).

After arriving at a comprehensive cytoprotective cocktail-based preconditioning to prevent peroxide-induced apoptosis in CDCs, the comprehensive preconditioning was tested to test whether it promoted cell survival post cardiac transplantation in an ischemia reperfusion-based setup. A rat model of myocardial ischemia reperfusion (IR) was used as a first grade model, and used CDCs transduced with luciferase expressing plasmid driven by CMV promoter.

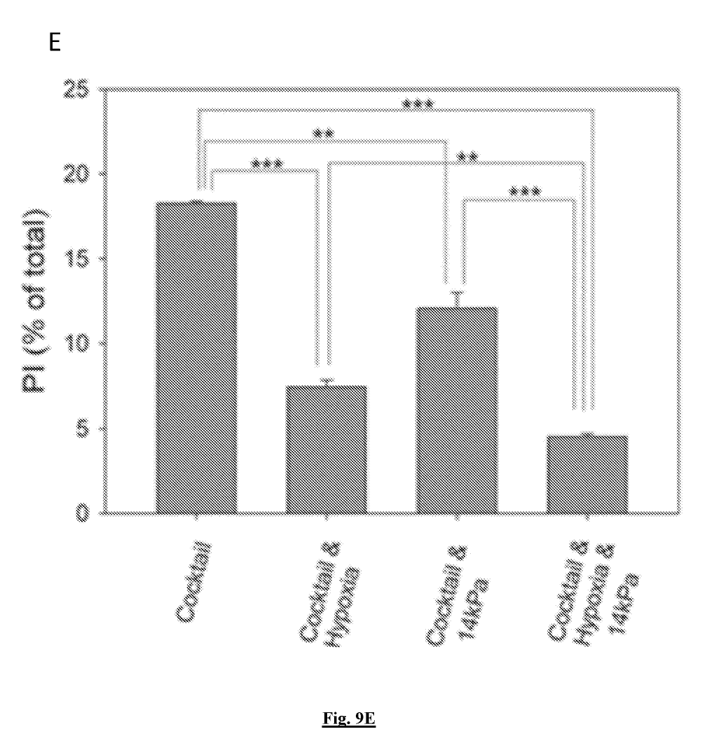

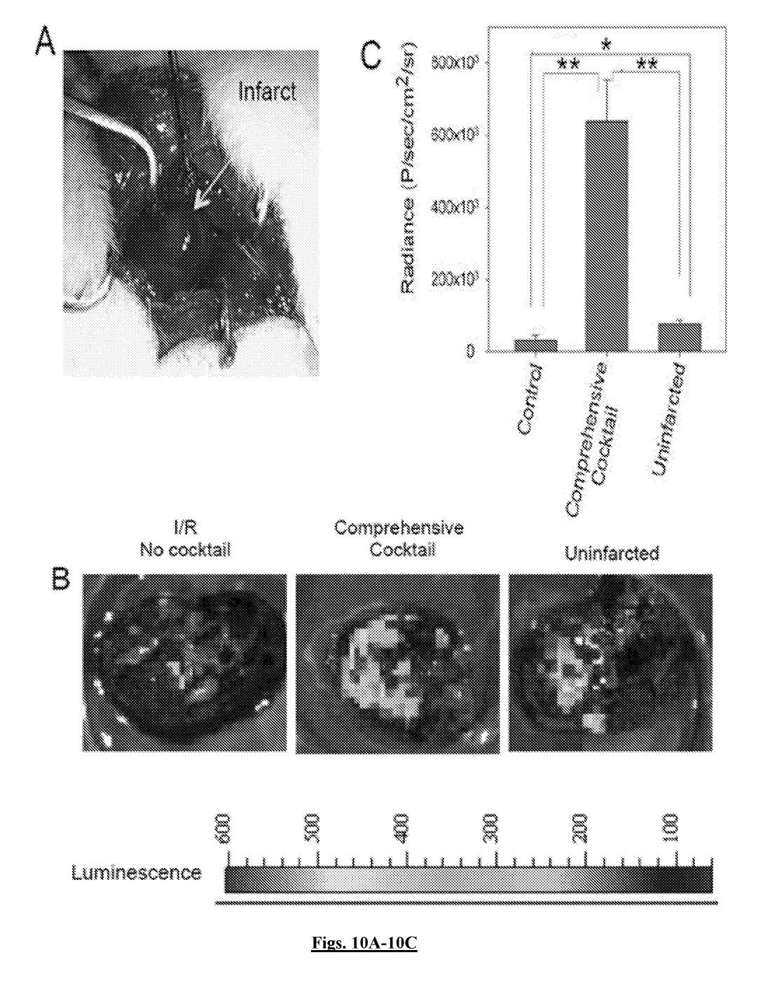

CDC-lv-luciferase were cultured on polystyrene surfaces, untreated, and in normoxia as controls, while the experimental groups were cultured on substratum with rigidity Y=14 kPa for 3 days, while preconditioned with 1% O₂, IGF-1, HGF, SDF-1α for 12 hours before trypsinization. Cells were injected at 2 sites bordering 2 days older infarcted zone in reperfused rat hearts (FIG. 10A), and bioluminescence measured after 36 hours in freshly isolated heart post peritoneal injection of D-luciferin (FIG. 10B). Bioluminescence revealed a significant increase in cell survival in uninjured heart as compared to controls, indicating that cells suffered significant death in control groups as compared to healthy hearts. CDCs preconditioned with comprehensive cocktail showed a significantly high survival rate as compared to control CDCs in FR injury model, but also remarkably, higher than control CDCs injected in healthy hearts (FIG. 10B-C).

### Example 2: μFLISA: Experimental and Computational Platform for Analysis of Dynamic Secretomes Uncovers a Secretion Signature Protecting Cardiac Cells from Reperfusion Induced Stress

To facilitate precise and absolute measurement of cellular secretion in arbitrary biological contexts, a platform combining protein microprinting and microfluidics was created. Microprinting of antibodies for sandwich immunoabsorbent assay allowed high throughput detection of cellular secretions, while microfluidic liquid handling allowed concentration of secreted ligands, obviating the need for enzymatic amplification. At the same time, placement of multiple detection spots for the same ligand at different distances away from the cells allowed for more precise evaluation of the diffusion of secreted molecules. These features were combined within the device and the associated method was termed “microfluidic fluorescence linked immunoabsorbent assay” (μFLISA) (FIG. 1A). μFLISA relies on the use of the initially separated experimental and a detection chambers. Cells are cultured in the experimental chamber, which is initially isolated from the detection chamber by valve-less method described more in detail below. This isolation allows for undisturbed cell adhesion and arbitrary pre-incubation experimentation with the cells, prior to initiation of secretion analysis. The detection chamber consists of rows of antibodies, each row consisting of different antibody species, with the potential of recognizing different proteins within the cell secretome (FIG. 1A).

The key element of the design of the device is the easy-to-control separation between the experimental and detection chambers, enabled by a row of closely positioned pillars (FIGS. 1A-1E). Prior to initiation of detection, there is no liquid in the detection chamber thus creating a liquid-air interface. The liquid-air interface and small distances between the pillars create considerable surface tension, serving to isolate the experimental chamber from the detection one. The surface tension can be overcome by an increase in the hydrostatic pressure in the experimental chamber, thus enabling gentle, laminar flow mediated ‘flooding’ of the detection chamber at the initial point of the detection (FIG. 1E).

Since the liquid in the experimental chamber can be exchanged prior to this point, the cellular medium can be devoid of accumulated cell secretions at the initial point of defection, allowing for more accurate analysis of the secretion kinetics. ComSol simulations (FIGS. 13A-13B) demonstrated that due to relatively small pillar size, ligand diffusion from the experimental to observation chamber is not hindered by pillars. The detection can be stopped at different time points, providing the information on the distribution of the accumulated secreted ligands in both space and time. These design elements allow μFLISA to detect the secretion by cells exposed to different biological conditions, potentially identifying correspondent unique secretory signatures as cellular phenotypes (FIG. 1B). In addition, the design includes three standardization chambers for on-chip calibration using known ligand concentrations (FIG. 1D).

First, the ability to control the initial point of detection of the secreted ligands was examined. In particular, cell adherence and spread prior to initiation of detection was tested. Since most adherent cells are phenotypically different in the suspended state vs. the state of full adhesion and spreading, it is important to allow cells to fully spread prior to initiation of detection. Furthermore, it may be important to permit these fully adherent cells to respond to stimuli of interest (e.g., treatment with drug, changes in oxygen tension, binding of a ligand), prior to detection of factors that may be conditioned on these additional inputs. BMSCs rapidly attached and spread, forming a complete monolayer in the experimental chamber (FIG. 1D).

The open design of the chamber allowed examination of the effect of normoxic and hypoxic (1% oxygen) conditions, with the pre-exposure to these conditions lasting 12 hours. BMSC secretion was then checked for the presence of cytoprotective factors previously implicated as important parts of the secretome of these cells: HGF, VEGF, IGF-1, DKK1, SDF-1α, and IL-6. These factors were focused on as potential mediators of the therapeutic effect associated with these cells. Absolute concentrations of the secreted factors at the detection spots closest to the cell populations were established using on-chip standards (FIGS. 13C-13D).

Interestingly, secretion profiles of BMSCs were significantly different in normoxia vs. hypoxia; while BMSCs secreted high absolute amounts of all six measured cytoprotective proteins in normoxia, they exhibited increased DKK1 secretion, and a decrease in HGF, IL6 and IGF1 secretions in hypoxia (Figure IH). This measurement was validated with flow cytometry analysis of the cells exposed to the same stimulation protocols, and the results were highly consistent (Figure IG). Surprisingly BMSCs did not increase VEGF secretion in response to hypoxia, though this may be because BMSCs might have a very limited basal capacity to translate and secrete VEGF. These data suggest that secretion profiles of BMSCs are highly sensitive to the presence of hypoxia, which could define differential response of these cells to natural or pathological alteration in the local oxygen levels. μFLISA platform also allowed absolute detection of HGF (FIGS. 4A-4B), SDF-1α (FIGS. 4C-4D) and DKK1 (FIGS. 4E-4F) after onset of normoxia or hypoxia (1% oxygen) for 6 hrs, 12 hrs, and 18 hrs. μFLISA measurements demonstrate that the detection chamber is not well-mixed, and the concentrations of detected ligands alter with changing distance from the experimental chamber (FIG. 4A).

The algorithm and the experimentally detected spatial distributions of several ligands at detection spots were tested at three different time points. More specifically, the secretory profiles of a few key cytoprotective factors were estimated: SDF-1α, HGF, and DKK1 after BMSCs were subjected to hypoxia (1% oxygen), or normoxia. μFLISA measurements were made 6 hours, 12 hours, and 18 hours after the cell microenvironment was altered, and the temporal kinetics were reconstructed using the above algorithm. The model predicted the detailed temporal secretion profiles, as well as recomputed the μFLISA-detected spatial intensity profiles, which was used for internal validation.

Since the model fit was required for three spatially graded profiles (at different time points), each composed of 12 independent data points, the procedure was highly restrictive on the fit parameters, and did not lead to over-fitting. Since analytical estimation of diffusion coefficients is difficult, as they may depend on a variety of parameters, including the molecule weight, hydration, viscosity of the medium, temperature, interaction with other molecular species etc., the computed solutions were fitted to the experimental data by varying the diffusion coefficient, and selecting the value producing the best fit. The algorithm produced well-fitting solutions for the SDF-1α (SSE=0.099 for hypoxia, 0.054 for normoxia), and HGF secretions (SSE=0.035 for hypoxia, 0.061 for normoxia).

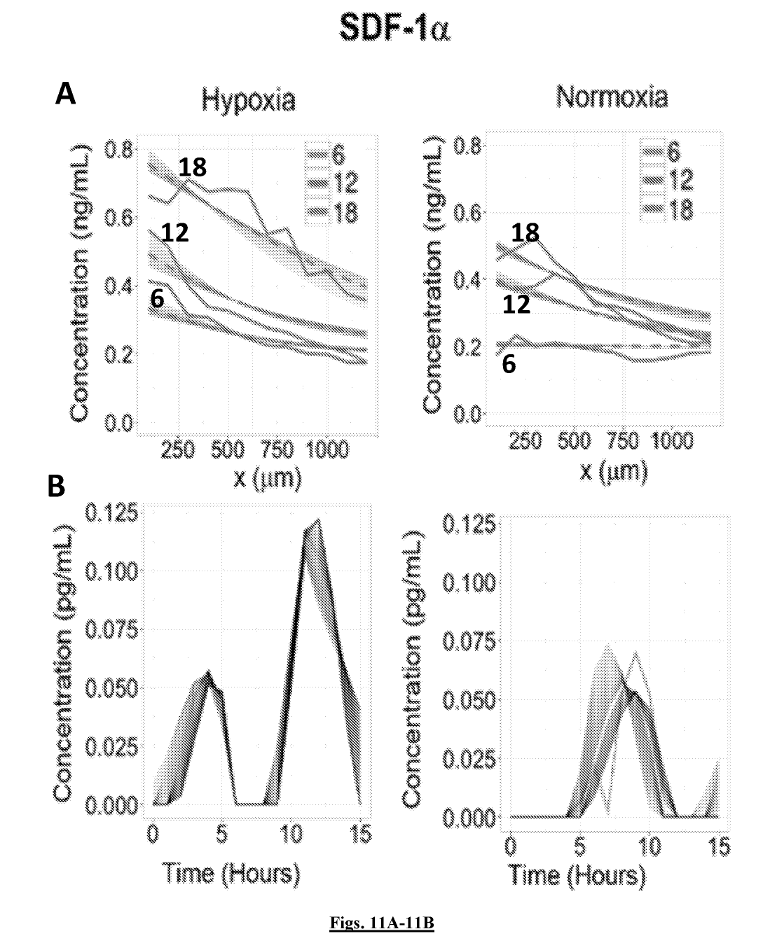

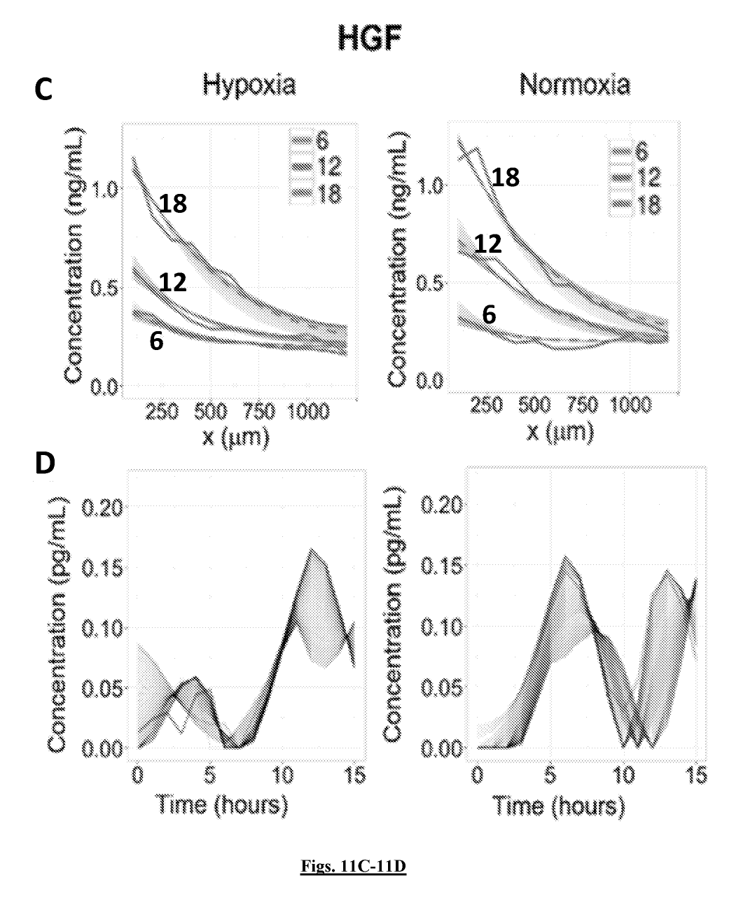

To account for experimental variation, a family of solutions was generated. Varying the values of the diffusion coefficient by 50% around the optimal fit values showed that the predicted kinetics were robust and could capture the essential temporal profiles of secretion (FIGS. 11A-11D). Furthermore, again, the temporal profiles of the probed cytokines showed differences between hypoxia and normoxia, suggesting that both the absolute values and dynamics of cell secretome can be affected by low oxygen. In particular, the algorithm inferred that BMSCs under hypoxic conditions secreted SDF-1α and HGF in two rapid successive pulses, peaking between 0 and 5 hrs, and 10-15 hrs.

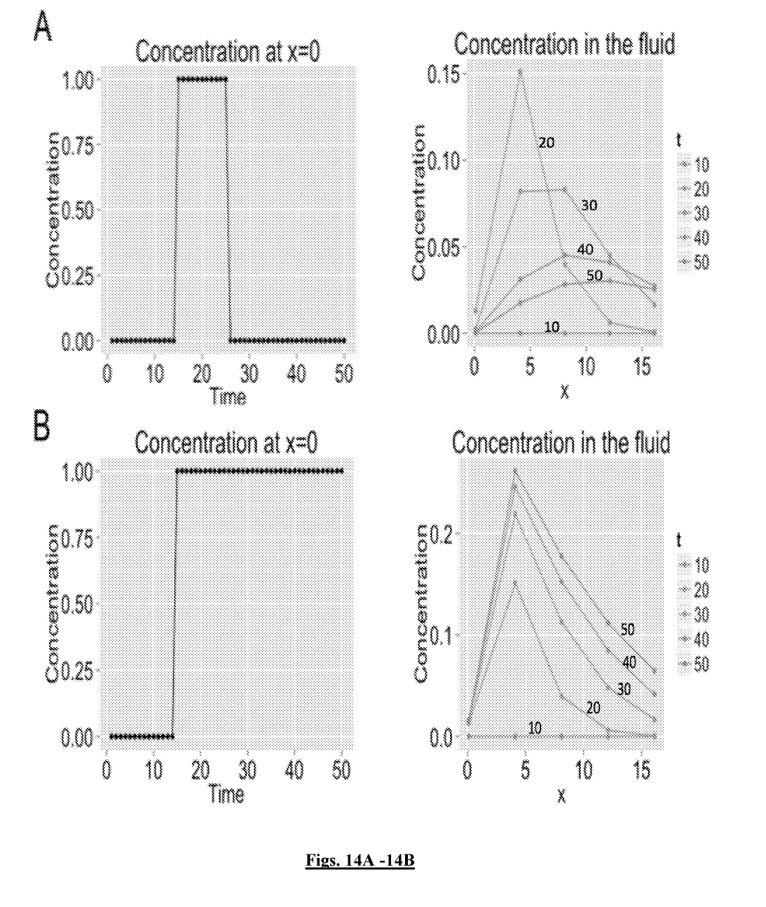

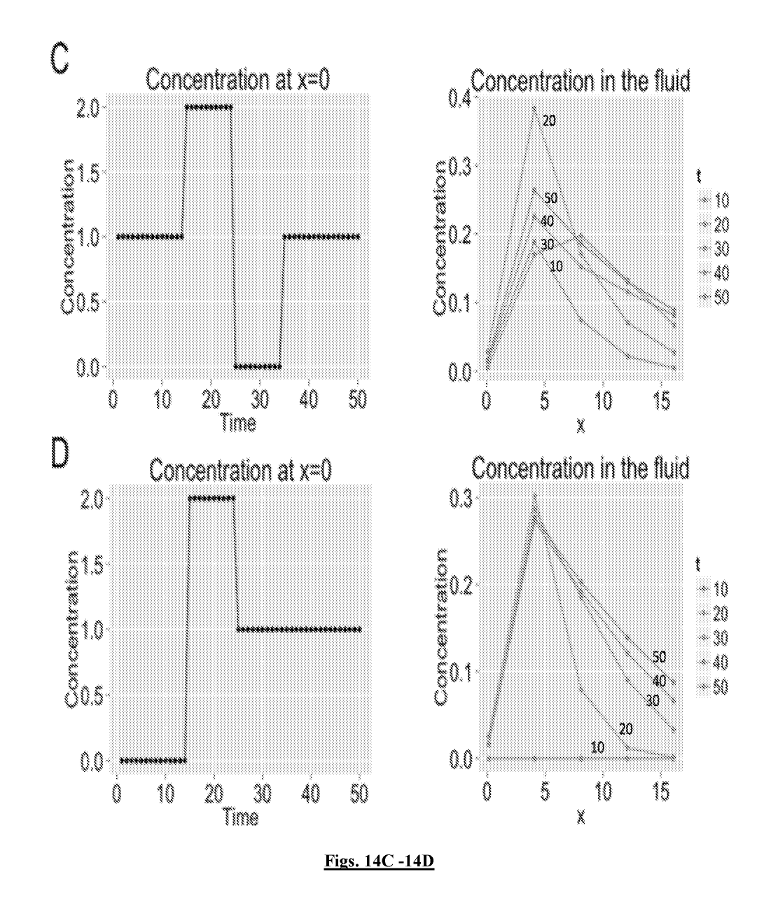

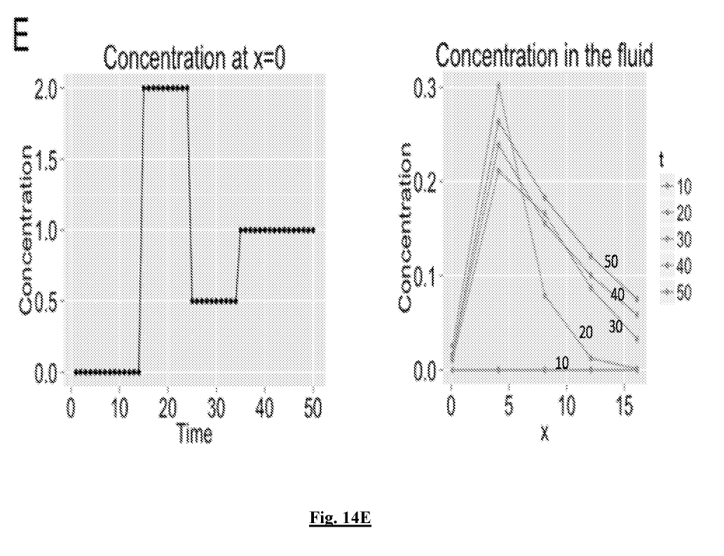

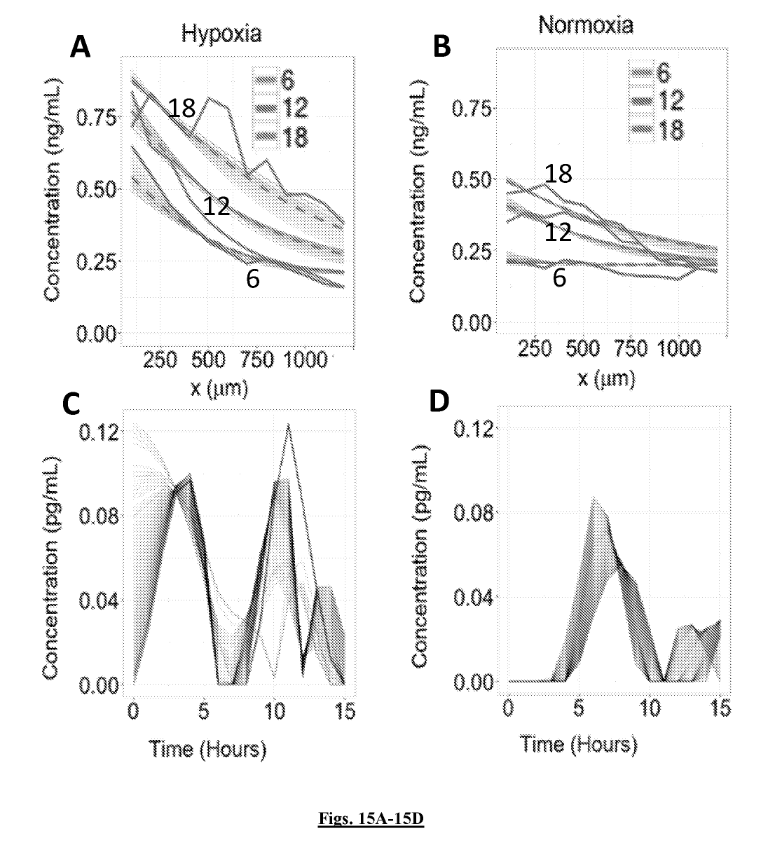

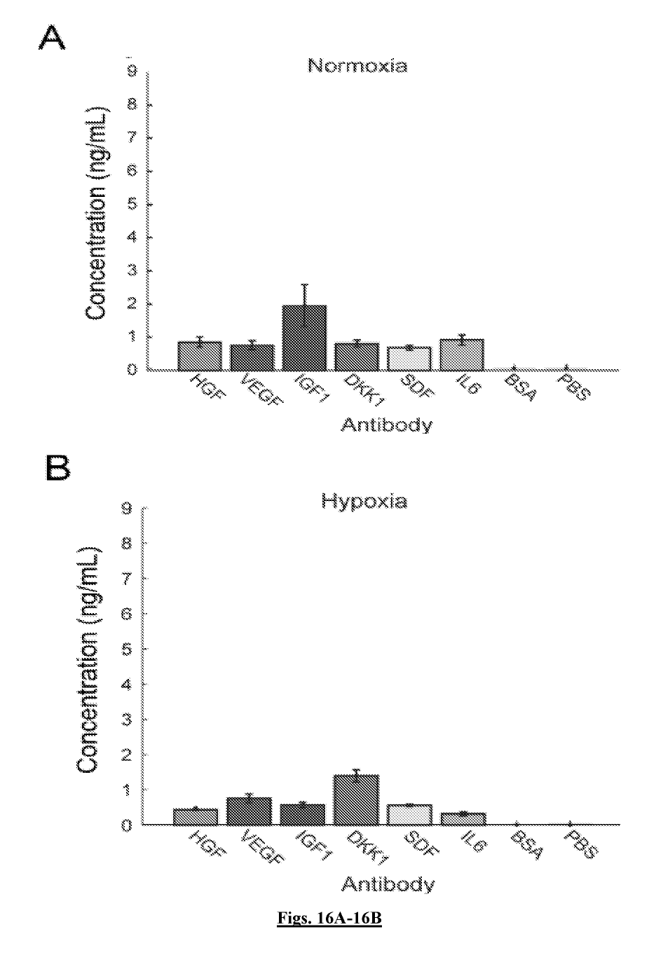

On the other hand, the secretion of these factors was delayed in normoxic conditions, with just one pulse fully observed over the first 15 hrs, peaking between 5 and 10 hrs after initiation of detection. Similar results were obtained for secretion of DKK1, although the fits were considerably worse, due to higher experimental variability (FIGS. 14A-14D). Overall, μFLISA platform could indeed predict detailed temporal kinetics of the secreted substances based on a very limited experimental time resolution.

A variety of stem cells have been reported to limit damage due to injury without underdoing direct differentiation into host tissue cell types. Therefore, there is increasing evidence that stem cells might beneficially affect the injured host tissue by paracrine signaling. However, how stem cells alter their secretory signatures in different biological contexts, particularly in response to injury signals, is not well known. If precise and absolute secretions of stem cells in response to injury can be determined, it would be possible to reconstitute the therapeutic effects of stem cells through a cell-free input, using a recombinant protein cocktail, whose composition would mimic the secretion profile of stem cells.

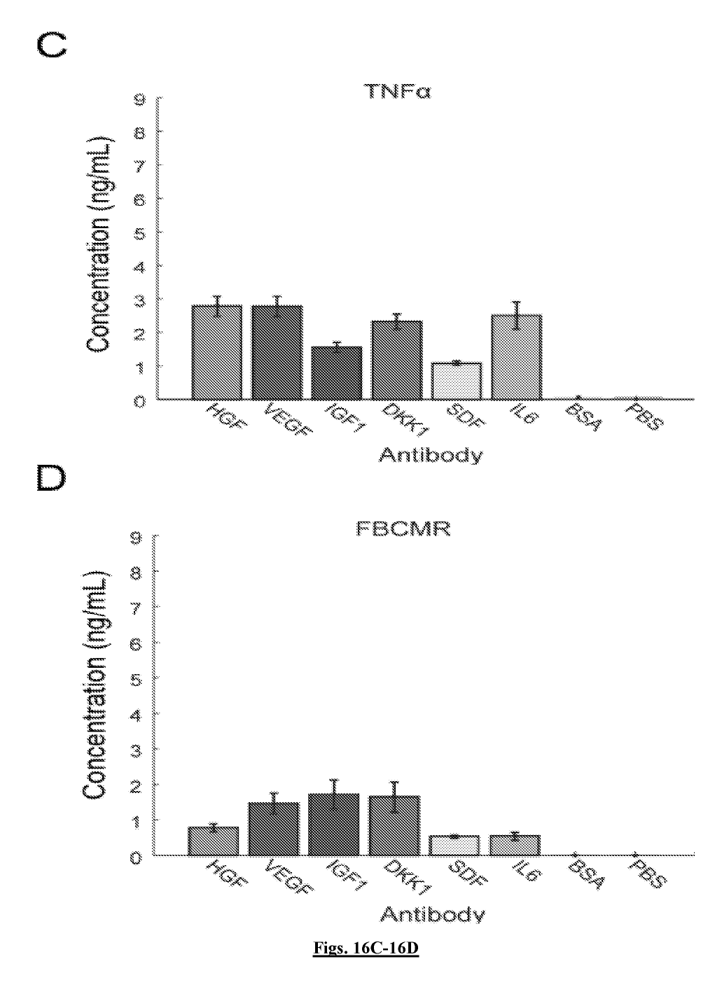

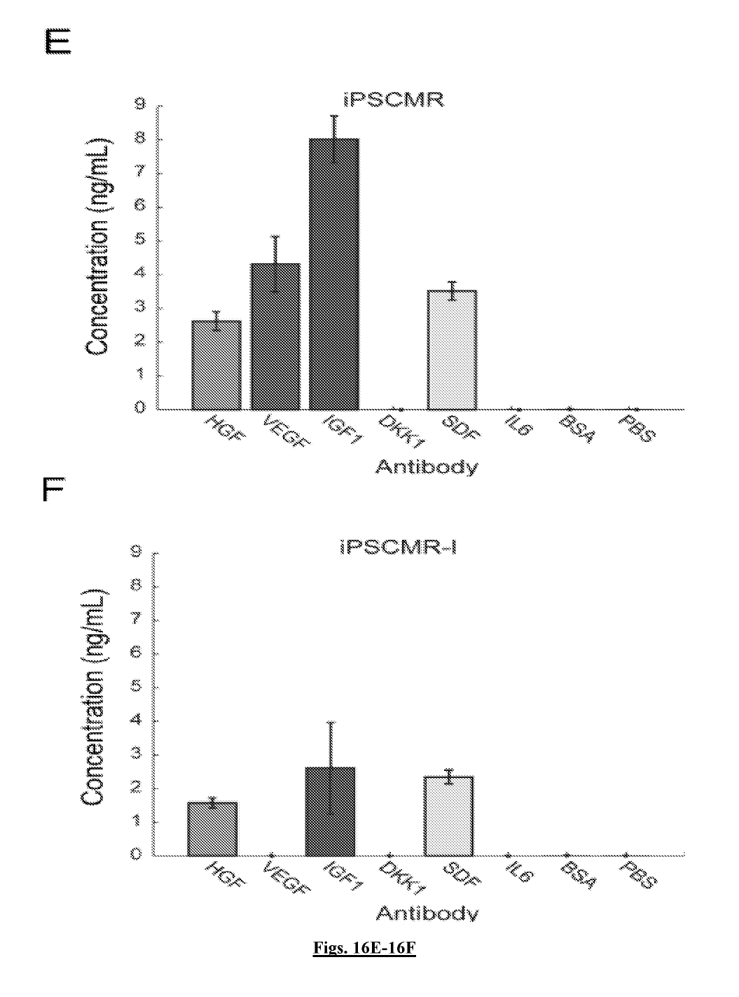

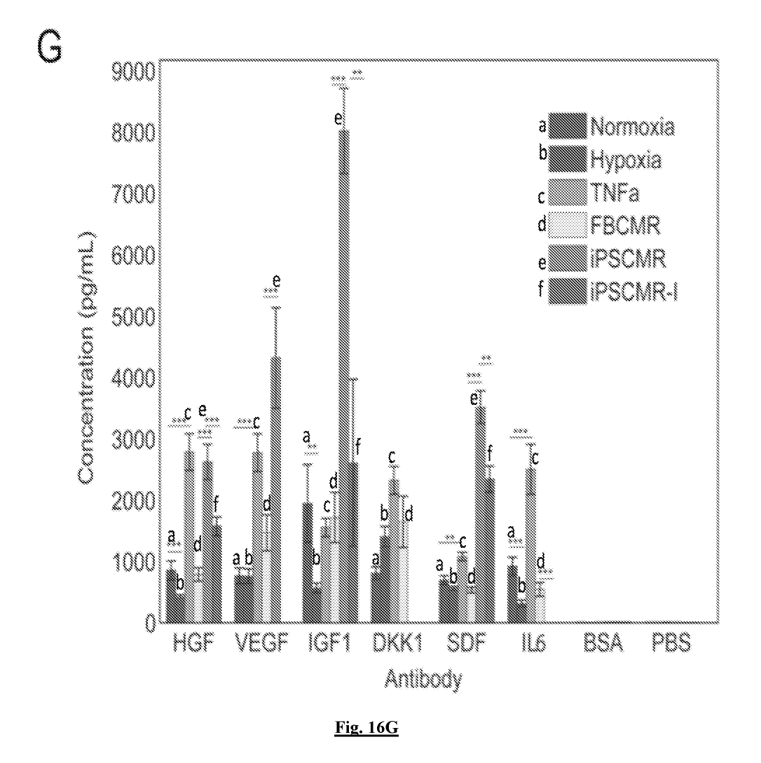

It is not, however, sufficient merely to determine the basal secretory signature of stem cells, including BMSCs, but also to be able to measure their secretions in response to various physiological stimuli that they typically respond to in vivo. Whether BMSCs could adjust the secretion profiles to various stimuli that could be present at the site of injury (including e.g., myocardial infarct39) was tested (FIGS. 5A-5C and 16). The highest measured values detected at the microspots closest to the secreting cells, at 18 hrs. of stimulation, were used for the analysis.

Both hypoxia and stimulation with a pro-inflammatory cytokine TNFα significantly altered the secretions of the molecular species investigated, but in a divergent manner. Whereas hypoxia had little effect on secretion of VEGF, the effect of TNFα on secretion of this factor was much more pronounced. On the other hand, whereas the effect of TNFα on secretion of IGF-1 was undetectable, there was a strong downregulation of this factor by hypoxia. For HGF, IL-6 and to a lesser degree, SDF-1α, there was a significant down-regulation of these factors by hypoxia and up-regulation by TNFα. Thus hypoxia and TNFα, both present at the site of many injuries, can have dissimilar effect on BMSC secretion, indicating the ability of the cells to adjust the response to the particular extracellular environment. Importantly, changes in secretion of some of the factors analyzed (e.g., SDF-1α) were minor in both conditions, suggesting that it is important to analyze a larger battery of secreted components to explore the ability of a cell to recognize and uniquely respond to complex changes in its microenvironment.

A more specific injury environment, mimicking the conditions accompanying myocardial infarction, was then modeled. Since it is difficult to individually isolate each biochemical factor present in the complex environment conditioned by myocardial infarction (MI), a model for MI using injured human pluripotent stem cells derived cardiomyocytes (iPSC-CMs) was created. To investigate the secretions of BMSCs in response to MI-like cell damage, BMSCs were conditioned with the medium collected from iPSC-CMs treated with Imatinib, an oxidative stress inducer. As controls, the cells were conditioned with the media collected from intact (un-insulted) iPSC-CMs, and unstimulated cardiac fibroblasts (FBCMR). μFLISA analysis of BMSCs stimulated by with the FBCMR conditioned medium (FIG. 5D) exhibited a similar profile to that produced by the control untreated BMSCs (FIG. 16). However, the secretory signature of BMSCs generated in response to the un-insulted iPSC-CMs conditioned medium changed drastically, showing no detectable DKK1 levels, but significant multi-fold increases in HGF, VEGF, IGF-1, and SDF-1a secretions (FIG. 5E). Surprisingly, BMSCs treated with medium conditioned by insulted iPSC-CMs exhibited a less dramatic change in the secretory signature, displaying no detectable VEGF, DKK1 and IL-6, but continuing to have increased levels of HGF, SDF-1α and high levels of IGF-1 (FIG. 5F). These results indicate that the secretion profiles of BMSCs can show strong condition specificity reflective of the nature of the neighboring cells and the extent of their stress or damage (FIG. 16G).

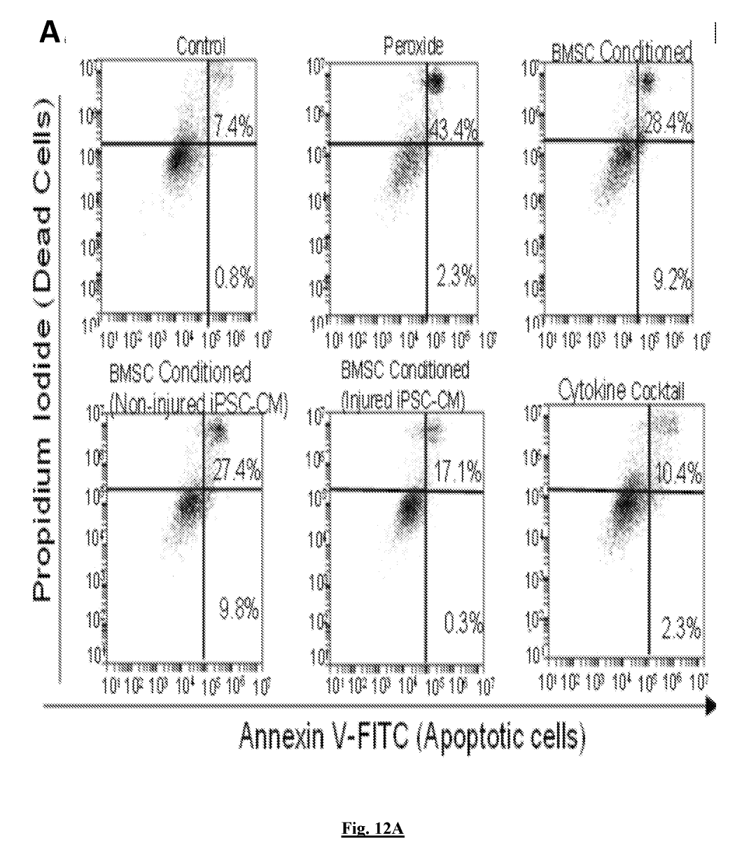

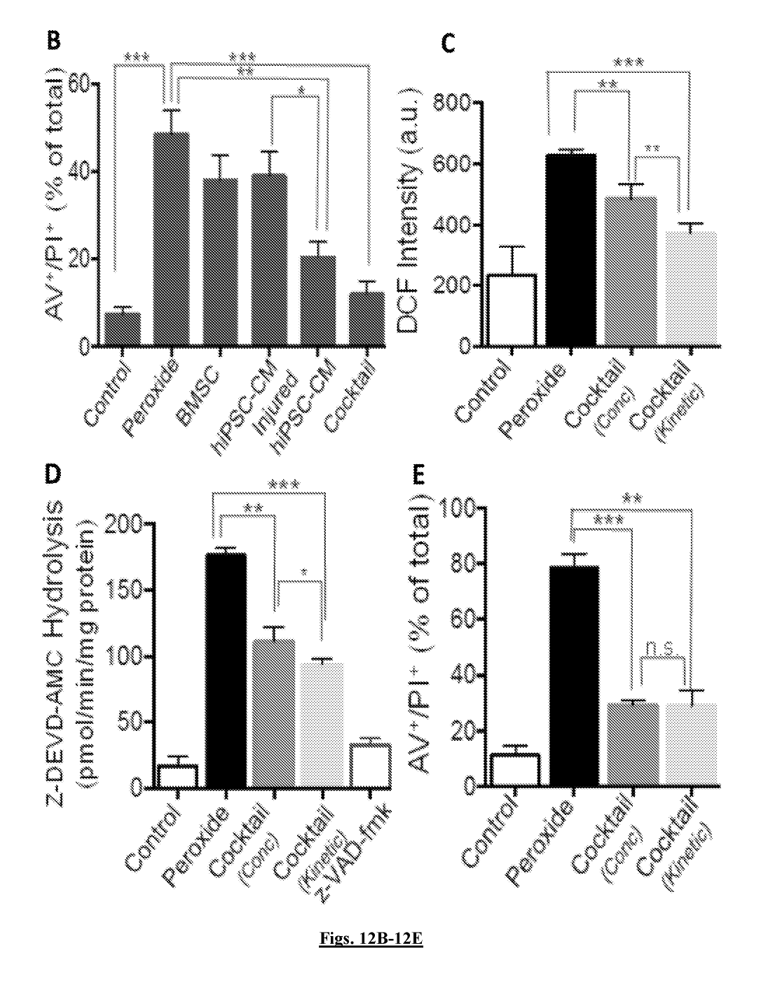

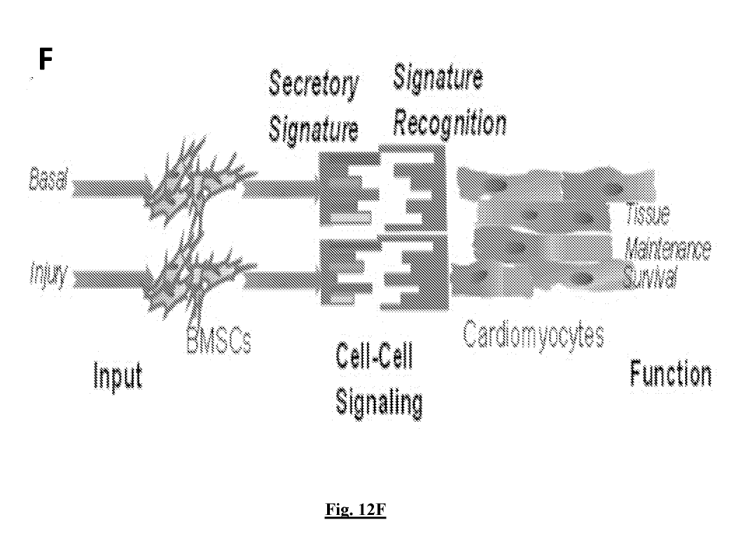

Since secretions from BMSCs are known to be cytoprotective at the site of MI in vivo, it was determined whether the cocktail of factors secreted by BMSCs in response to the medium conditioned by insulted iPSC-CMs could also assist in rescuing these model cardiomyocytes from reperfusion induced apoptosis. Human iPSC-CMs were cultured as a beating monolayer and the extent of apoptosis was measured after treatment with 500 μM H2O2 for 30 minutes in the presence of BMSC conditioned medium using Annexin V/PI apoptosis assay (FIG. 12A). BMSCs were themselves pre-treated in ways mimicking MI and ischemia reperfusion (FR) micro-environments, as described above. BMSC conditioned medium without any specific additional cell pre-conditioning could potently reduce peroxide induced death of hiPSC-CMs. This beneficial effect was further dramatically increased when BMSCs were additionally pre-treated with the medium conditioned by injured iPSC-CMs (FIGS. 12A-12B). These data strongly support the hypothesis that stem cells in general, and BMSCs in particular, can generate contextual paracrine signaling consisting of a potent cytoprotective secretory signature in response to an injury signal.

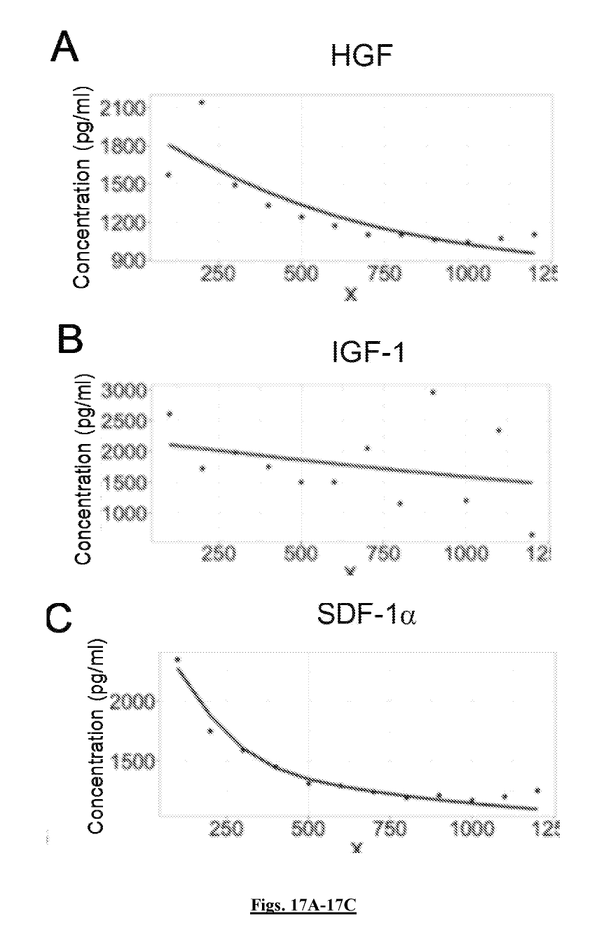

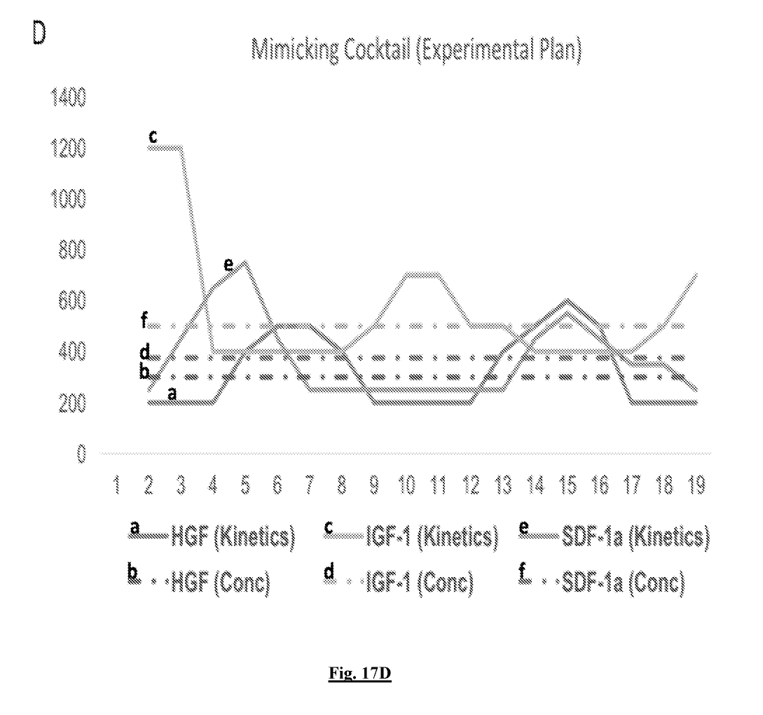

If the cocktails of factors secreted by BMSCs in response to the signaling from the injured iPSC-CMs, as identified by μFLISA, mediate most of the cytoprotective activity of the BMSCs conditioned medium, one can attempt to reproduce this activity using recombinant factors. Thus, the effect of the recombinant factors combined in concentrations matching the average experimentally determined values was examined (FIG. 5F). Strikingly, it was found that this cocktail had a potent cytoprotective effect closely matching the effect of the BMSCs conditioned medium (FIGS. 12A-12B). It was then determined whether matching the dynamics of individual factors constituting the cocktail could further alleviate peroxide induced stress in hiPSC-CMs. Using the algorithm, the secretion dynamics of each individual factor present in the cocktail were estimated (FIGS. 17A-12D). Then, the injured hiPSC-CMs were conditioned with the media containing the dynamically varying factor inputs in the cocktail, whose time-integrated values matched the average values in FIG. 5F. Media were changed every 1 hour to maintain the dosage dynamics (FIG. 17D). Intracellular ROS levels in hiPSC-CMs after peroxide treatment were significantly lowered following cell exposure to dynamically delivered cocktail vs. statically delivered cocktail with average factor concentrations (FIG. 12C). Furthermore, caspase 3 activity as measured by hydrolysis of DEVD was significantly lower in hiPSC-CMs treated with the dynamically varying cocktail (FIG. 12D). No significant difference was found between dynamically and statically delivery protocols in the assays relying on the integrity of the plasma membrane as measured by Annexin V/PI assay, suggesting that the primary effect of cocktail dynamics is primarily in caspase and ROS dependent processes (FIG. 12E). These data highlight that not only the average secretion, but also the dynamics of cellular secretion conveys unique signal to the detecting cells to respond, here by reducing their redox stress levels and decreased apoptosis. This result suggests that precise and absolute measurements of cellular secretions, and their dynamics in response to physiologically relevant stimuli can allow creation of cell-free therapy modalities.

The disclosures of each and every patent, patent application, and publication cited herein are hereby incorporated herein by reference in their entirety.

While the present invention has been disclosed with reference to specific embodiments, it is apparent that other embodiments and variations of the present invention may be devised by others skilled in the art without departing from the true spirit and scope of the invention. The appended claims are intended to be construed to include all such embodiments and equivalent variations.

What is claimed is:

1. A device for the temporal high-throughput measurement of one or more molecules or compounds secreted by a cell using quantitative enzyme linked immunosorbant assay (qELISA), the device comprising an experimental chamber and an observational chamber, wherein the experimental chamber and the observational chamber are separated by a permeable barrier, wherein the permeable barrier is selected so that movement of the one or more molecules or compounds across the permeable barrier is hindered when the observational chamber comprises air and/or is free of liquid.

2. The device of claim 1, wherein the device further comprises one or more standardization chambers, one or more experimental chambers, and/or one or more detection chambers.

3. The device of claim 1, wherein the experimental chamber allows the adhesion of the cell.

4. The device of claim 1, wherein the observational chamber comprises rows of molecule or compound detection location, wherein each row is arranged transversely to the experimental chamber and comprises an antibody that selectively binds a biological molecule.

5. The device of claim 1, wherein the one or more molecules or compounds secreted by the cell migrate from the experimental chamber to the observational chamber through diffusion based movement.

6. A method of calculating an intensity of a cellular secretion using the device of claim 1, the method comprising:
a) contacting cells with the experimental chamber;
b) exposing the cells to experimental conditions to induce secretion of the one or more molecules or compounds;
c) moving the one or more molecules or compounds from the experimental chamber into the observation chamber;
d) binding the one or more molecules or compounds to one or more molecule or compound detection locations in the observational chamber; and
e) calculating an intensity of the one or more molecules or compounds.

7. A method of generating a temporal intensity profile of one or more molecules or compounds secreted from a cell, the method comprising:
a) calculating an estimated intensity of the one or more molecules or compounds at a distinct molecule or compound detection location and time based on diffusion of the one or more molecules or compounds to the detection location (g[x,t]);
b) calculating an observed intensity at the detection location due to an adsorption and binding of the one or more molecules or compounds to the molecule or compound detection location at an observed time (s[x,t]);
c) calculating a difference between b) and a) (s[x,t]−g[x,t]) to obtain a loss function;
d) updating the estimated intensity to minimize the loss function;
e) generating the intensity profile for the one or more molecules or compounds at the molecule or compound detection location; and
f) repeating steps a) through e) for a plurality of molecule or compound detection locations,
thereby training a function minimization algorithm to generate the temporal intensity profile of the one or more molecules or compounds secreted from a cell.

8. A method of generating a temporal concentration profile of one or more molecules or compounds secreted from a cell, the method comprising:
a) calculating an estimated concentration of the one or more molecules or compounds at a distinct molecule or compound detection location and time based on diffusion of the one or more molecules or compounds to the molecule or compound detection location (c[t]);
b) proposing a deviation (d[t]) from the estimated concentration (c[t]+d[t]);
c) calculating an observed concentration at the molecule or compound detection location due to an adsorption and binding of the one or more molecules or compounds to the molecule or compound detection location at an observed time (s[x,t]);
d) calculating a difference between b) and c) (c[t]+d[t]−s[x,t]) to obtain a posterior probability of the deviation;
e) accepting or rejecting the proposed deviation of d[t] based on the ratio of the posterior probability of (d) compared to the estimated concentration a);
f) generating the concentration profile for the one or more molecules or compounds at the molecule or compound detection location; and
g) repeating steps a) through f) for a plurality of molecule or compound detection location,
thereby training a function minimization algorithm to generate the temporal concentration profile of the one or more molecules or compounds secreted from a cell.

9. A method of detecting a secretion, and/or level of secretion, of a molecule or compound by a cell isolated from a subject, the method comprising measuring and determining temporal intensity profile and/or temporal concentration profile of the molecule or compound using the device of claim 1.

10. The method of claim 9, wherein the subject is a mammal.

11. The method of claim 9, wherein the cell is an adherent cell selected from the group consisting of fibroblasts, immune cells, cancer cell lines, primary cancer cells, stem cells, progenitor cells, stromal cells, pluripotent stem cells, somatic cells derived from pluripotent stem cells, and somatic cells derived from adult stem cells.

12. The method of claim 9, wherein the cell is a non-adherent cell.

13. The method of claim 9, wherein the cell is derived from healthy or diseased heart tissue, connective tissue, vasculature, brain tissue, tumor environment and/or metastatic tumor environment.

14. The method in claim 9, wherein the cell is derived from a tissue explant that is placed in the experimental chamber from healthy or diseased heart, vasculature, brain, tumor, liver, pancreas, spleen, bone marrow, cartilage, adipose tissue, and/or connective tissue.

15. The method of claim 9, wherein the cell is pretreated by a stimulus.

16. The method of claim 15, wherein the stimulus comprises at least one from the group consisting of a drug, cytokine, growth factor, hypoxia, pathogen load, physical, chemical, mechanical, and biological stimulus.

17. The method of claim 9, wherein the cell is cultured in a biologically mimicking environment.

18. The method of claim 9, wherein the cell is co-cultured in a system selected from the group consisting of cancer cell in the presence of immune cells, immune cell in the presence of cancer cells, stem cell in the presence of immune cells, stem cell in the presence of stromal cells, stromal cell in the presence of stem cells, endothelial cell in the response to cancer cells, cancer cell in the response to endothelial cells, and cancer cell in the presence of other cancer cells.

19. A method of identifying a cell isolated from a subject, the method comprising measuring and/or determining a temporal intensity profile and/or temporal concentration profile of one or more molecules or compounds using the device of claim 1, wherein the profiles identify at least one selected from the group consisting of cell type, cell state, and cell response to a biological stimuli.

20. The method of claim 18, wherein the cell state comprises cell signaling, cell fate, cell age, and/or cell cycle.

21. A method of treating a disease or disorder in a subject in need thereof, wherein the treatment is cell-free, the method comprising the steps of:
a) identifying a first temporal intensity profile and/or temporal concentration profile for one or more molecules or compounds secreted by a cell that is used for treating the disease or disorder, wherein the first profiles comprise one or more biological molecules,
b) identifying a second temporal intensity profile and/or temporal concentration profile for one or more molecules or compounds secreted by various cell types used to treat the same disease or disorder, wherein the second profiles comprise one or more biological molecules, and
c) administering to the subject a therapeutically effective amount of the one or more molecules comprised in either the first or the second profiles, wherein the subject is not administered a therapeutically effective amount of the cell.

22. The method of claim 20, wherein the cell comprises at least one selected from the group consisting of stem cell that secretes anti-apoptotic factors, stromal cell that secretes multipotency or differentiating factors, immune cell that secretes chemokines that inhibit cancer, immune cell that secretes chemokines that support cancer invasion secreted, and cancer cell that secretes a chemokine that promotes angiogenesis.

23. A method of identifying post-translational modification of secreted molecules from a cell in a specific biological condition, the method comprising measuring and determining the kinetics and temporal profiles of a cell exposed to a specific biological condition using the device of claim 1.

24. The method of claim 23, wherein the modification is selected from the group consisting of glycosylation, salicylic acid decoration, splicing, polymerization and other post translational modifications.

25. A composition comprising one or more growth factors selected from the group consisting of VEGF, SDF-1α, FGF8, IGF1, insulin, HGF, EGF, IGF1, and SCF, wherein the composition provides cytoprotection and prevents cellular apoptosis when contacted with a cell.

26. The composition of claim 24, wherein the composition comprises IGF1, HGF and SDF-1α.

27. The composition of claim 24, wherein the composition is used to treat or prevent cardiac injury.

28. The composition of claim 24, wherein the cytoprotection is against peroxide.

29. A method of treating a disease or disorder in a subject in need thereof, the method comprising administering to the subject a composition comprising one or more growth factors selected from the group consisting of VEGF, SDF-1α, FGF8, IGF1, insulin, HGF, EGF, IGF1, and SCF.

30. The method of claim 28, wherein the composition comprises IGF1, HGF and SDF-1α.

31. The method of claim 28, wherein the disease or disorder comprises cardiac injury.

32. A composition comprising one or more molecules, wherein the composition preconditions cells with mechanical and hypoxic preconditioning to induce a desired response.

33. The composition of claim 30, wherein the response comprises cell survival, prevention of cell proliferation, cell differentiation, cell multi- or pluri-potency, cell migration, and other cellular phenotypes.
